(19) United States
(12) Patent Application Publication
(10) Pub. No.: US 2016/0058324 A1
(43) Pub. Date: Mar. 3, 2016

(54) ULTRA LOW POWER CHARGING IMPLANT SENSORS WITH WIRELESS INTERFACE FOR PATIENT MONITORING

(71) Applicant: ARIEL CAO, OAKLAND, CA (US)

(72) Inventor: ARIEL CAO, Oakland, CA (US)

(21) Appl. No.: 14/789,942

(22) Filed: Jul. 1, 2015

Related U.S. Application Data
(60) Provisional application No. 62/044,895, filed on Sep. 2, 2014.

(51) Int. Cl. A61B 5/07; A61B 3/00; A61B 5/00; A61B 3/16

(52) U.S. Cl. CPC A61B 5/076; A61B 3/16; A61B 3/0025; A61B 5/4836; A61B 5/746; A61B 5/7282; A61B 2560/0219; A61B 2562/085

(57) ABSTRACT

Methods and devices for monitoring intra-ocular pressure of a patient using a miniature implantable sensor device are provided herein. Methods include obtaining multiple pressure measurements each day during an increment of an extended monitoring period according to a sampling program and wirelessly transmitting stored measurement data and wirelessly charging the device. Measurements and data process is performed with low power requirements such that sampling can be performed hourly for at least one week using energy stored on the miniature device and measurement data can be transmitted and the device charged rapidly when an external portable data acquisition/charging device is held in proximity to the device. In one aspect, methods include switching between differing use modes and powering the sampling device with a high impedance battery by switching between a supercapacitor and the battery with a microcontroller to perform impedance conversion.

### CROSS-REFERENCES TO RELATED APPLICATIONS

The present application claims the benefit of priority of U.S. Provisional Patent Application Nos. 62/019,826 filed Jul. 1, 2014; 62/019,841 filed Jul. 1, 2014; and 62/044,895 filed Sep. 2, 2014; each of which is incorporated herein by reference in its entirety.

The present application is related to the following co-assigned applications: U.S. Provisional Patent Application Ser. No. 62/019,826 (Attorney Docket No. 96933-000100US) entitled “Methods and Devices for Implantation of Intraocular Pressure Sensors” filed on Jul. 1, 2014, U.S. Provisional Patent Application Ser. No. 62/019,841 (Attorney Docket No. 96933-000200US) entitled “Hermetically Sealed Implant Sensors with Vertical Stacking Architecture” filed on Jul. 1, 2014, concurrently filed U.S. Non-Provisional patent application Ser. No. 14/789,491 (Attorney Docket No. 96933-000110US-947259), and concurrently filed U.S. Non-Provisional patent application Ser. No. ______ (Attorney Docket No. 96933-000210US-947262); each of which is incorporated herein by reference in its entirety for all purposes.

### BACKGROUND OF THE INVENTION

This application relates generally to devices and methods for monitoring intraocular pressure (IOP) within an eye of a patient, in particular, methods of sampling IOP with a miniature implanted device. Aspects include improve power management and/or configurations with ultra low power requirements that allows continuous frequent monitoring without patient initiation and only periodic rapid charging and telemetry.

Glaucoma is a condition resulting in increased pressure within the eye that eventually leads to damage of the optic nerve that transmits images to the brain, which results in gradual vision loss. The increased pressure within the eye causes a loss of retinal ganglion cells in a characteristic pattern of optic neuropathy. A patient suffering from glaucoma typically experiences a build-up of aqueous fluid which increases the pressure inside the eye (i.e. IOP). Elevated IOP is one of the primary risk factors for developing glaucoma, which must be carefully monitored and controlled in treating glaucoma. As retinal ganglion cells are damaged by glaucoma, the visual signals from at least a portion of visual field are no longer reported to the brain, forming blind spots or scotomas. As glaucoma progresses and increasingly damages more nerve tissue in the optic nerve, vision loss continues as the scotomas increase in size and/or number. Failure to properly treat glaucoma and to reduce and monitor the IOP may cause irreversible vision loss. Untreated glaucoma, which affects one in 200 people under the age of fifty and 10% of those over the age of 80, is the second leading cause of blindness worldwide. As of 2012, about 60 million people suffer from glaucoma world-wide and it is estimated that, by 2020, about 80 million people will suffer from glaucoma. In addition, since a high percentage of people are over the age of 75 years old, and as the world-population ages and life-spans increase, it is expected that glaucoma patient populations will continue to increase.

IOP in a healthy human eye is generally between 10 mmHg and 20 mmHg. Glaucoma causes substantial increase in and/or variation in IOP than that experienced in a healthy eye. The IOP is determined largely by the amount of aqueous fluid entering and exiting the eye. Aqueous fluid is produced by the ciliary body to supply the lens and cornea with nutrients and carry away waste products. Normally, aqueous fluid flows between the iris and the lens, through the pupil and to the drainage angle before exiting the eye through a tissue called the trabecular meshwork in the drainage angle. If the aqueous fluid is produced at a rate faster than it drains, then the IOP will rise. An elevated IOP is associated with two major types of glaucoma: open-angle glaucoma and closed-angle glaucoma. In open-angle glaucoma, the drainage angle between the cornea and the iris is open and allows the aqueous fluid of the eye to reach the trabecular meshwork, but abnormalities in the trabecular meshwork reduce the outflow of aqueous fluid from the eye. In closed-angle glaucoma, obstructions within the trabecular meshwork prevent the aqueous fluid from draining properly out of the eye.

While the progression of glaucoma can be substantially halted in many patients using a variety of treatments, for example, medicines, prescription eye drops, shunts, and surgical procedures, failure to properly diagnose and/or monitor the IOP of a patient can drastically reduce the effectiveness of available treatments. Currently, glaucoma monitoring often uses infrequent IOP measurements obtained by a physician at a medical facility. For example, a typical patient may have their IOP measured on average four to six times per year by non-invasive techniques, such as tonometry. While tonometry techniques are generally low cost, easy, and non-invasive, a number of different types of errors can significantly reduce the accuracy of this diagnostic tool and as such potentially result in inappropriate diagnosis and/or ineffective follow-up medical treatment.

For example, at least some of these non-invasive clinical techniques may not detect elevated IOP levels (e.g., pressure spikes) as only a single point measurement is taken during an eye exam. Failure to continuously and/or frequently monitor IOP levels outside the eye clinic (e.g., more than four to six measurements per year) may lead to inaccurate detection of the patient's real IOP profile (e.g., real IOP may be higher or lower than measured IOP). Non-invasive measurements in some instances also lack accuracy as these devices measure pressure of the eye with an external sensor that provides an indirect measurement of the actual pressure inside the eye. For example, factors that affect accuracy may include failure to account for anatomical differences, such as a patient's cornea thickness, scleral rigidity, or conical curvature, variances due to operator's use or technique, physiological influences, such as caffeine or alcohol use, or prior refractive surgery that may affect a patient's IOP, etc. Hence, the indirect IOP measurements from such non-invasive devices may differ from the actual IOP inside the eye (e.g., overestimated or underestimated) which may lead to inappropriate diagnosis and/or follow-up treatment. Further, it often inconvenient and impractical for patients to visit the eye clinic on a strict regular schedule for repeated IOP measurements.

Although implantable IOP devices have been proposed for direct IOP measurements on a daily basis, these first generation implants may also suffer from several drawbacks which in turn may result in indirect and/or inaccurate measurement of IOP and inappropriate medical treatment of glaucoma. For example, the IOP devices may be too large or bulky in dimension, size or shape to be safely and effectively placed entirely within a desired location or structure of the eye for direct measurement of IOP. Further, some devices may be extremely invasive, requiring major surgery for implantation and/or complicated positioning of multiple components which are each implanted in different structures or areas of the eye, which unnecessarily increases patient risk and/or injury and total healthcare costs.

Further, some implantable devices for IOP measurement may utilize pressure ports which are susceptible to sensing inaccuracies or require direct implantation within certain anatomical locations, such as the anterior chamber, posterior chamber, suprachoroidal space, or cornea of the eye which may lead to unanticipated complications. Also, some of these devices may not be well suited for chronic implantation due to IOP implant design issues of water ingress and/or thermal stress (e.g., associated with polymer packaging), which in turn precludes continuous monitoring of IOP. Such proposed flexible sensors also have issued of degraded stability. In some instances, some IOP devices also suffer from poor calibration and/or monitoring is not adjustable so as to further result in inaccurate IOP detection levels.

Accordingly, it would be desirable to provide improved implant devices and methods of implantation that overcome at least some the above mentioned shortcomings. In particular, it would be desirable to develop miniature implantable IOP devices that provide more frequent sampling continuously over an extended monitoring period as well as adjustable sampling of IOP. Ideally, such devices should directly measure IOP levels and be safely and effectively implanted entirely within a desired location within the eye quickly and easily in an outpatient environment, such as the physician's office, without invasive major surgery. Such devices should further allow for chronic implantation so as to provide long-term stable and a continuous IOP measurement profiles for appropriate diagnosis and follow-up therapy. In addition, there exists a need for improved methods of implantation for such devices that allow for long-term monitoring of IOP with an implantable sensor without requiring surgical intervention and with minimal interaction by patient. There further exists a need for methods of monitoring IOP with reduced power consumption requirements and simplified charging and wireless transmission of measured data to allow for improved monitoring and patient compliance.

### BRIEF SUMMARY OF THE INVENTION

The invention provides devices and methods for obtaining IOP measurements within an eye of a patient multiple times a day with a miniature sensor device implanted within an eye and wirelessly charging the sensor device and communicating the IOP measurements to an external device or server for monitoring and/or trending of IOP measurements for improved glaucoma treatments. In one aspect, the invention provides a sensor device that measures and stores IOP data with ultra low power consumption such that the IOP measurements can be obtained more frequently, up to hourly sampling, and stored over a time increment of at least one week on a single charge of the miniature device, the single charge being performed in a relatively short duration of time, such as about three hours or less, typically between about 20 minutes and 2 hours depending on the number of charging cycles experienced by the device over its lifetime. In some embodiments, the sensor device includes a microcontroller that allows the sensor device to switch between differing use modes, allows for improved versatility and reprogramming/updating, as well as improved power management. For example, in some embodiments, the sensor device uses a microcontroller to power the sensor device functions with a high impedance thin-film battery by switching between the battery and a supercapacitor which decouples the battery from an ASIC of the device so as to perform impedance conversion. Such configurations allow for improved power management and increased functionality.

In one aspect, the present invention relates to monitoring of IOP by measuring pressure with a miniature sensor device implanted within the vitreous body and storing multiple pressure measurements on a memory of the sensor device each day during a time increment of an extended monitoring period according to a sampling protocol stored on the device memory. The sensor device periodically transmits the stored pressure measurements to an external data acquisition device using one or more coils adapted for wireless communication. The one or more coils may also be adapted for wireless charging of the device. In some embodiments, the one or more coils include a first coil adapted for wireless communication and a second coil adapted for wireless charging, which may be performed concurrently or sequentially when the external reader/charging device is held in close proximity to the implanted device. Alternatively, the sensor device may utilize a single coil adapted for both wireless communication and wireless charging, which may be performed sequentially according to a predetermined telemetry protocol. In one aspect, the sensor device is configured to communicate and/or charge at low RF power rates so that it may advantageously operate at ultra-low power requirements, such as 1 μW or less. Further, such ultra-low power requirements may not necessitate any particular alignment (e.g. rotational alignment) between the implantable sensor device and the external charger unless the recharging period is above the upper bound of 15 seconds (due to low power transfer efficiency or the limited allowable tissue exposure to the AC magnetic fields). This allows the miniaturized sensor device to charge and communicate easily and rapidly, typically within a few seconds or less, when the external data acquisition/charging device is held in various different positions so long as the external device is within a certain proximity, such as about 6 inches or less, typically within 2 inches or less of the sensor device.

Since the mechanisms contributing to the increase in intra-ocular pressure occur within the anterior chamber or adjacent thereto, conventional methods generally focus on measuring IOP within the anterior chamber. Because the anterior chamber is a particularly sensitive region, great care must be taken to avoid contacting the various parts of the anterior chambers, which may result in damage to the delicate structures therein. Since the pressure within the anterior chamber pushes against and increases the pressure within the vitreous body, measurement of pressure within the vitreous body provides a relatively accurate pressure measurement of IOP of the eye. In certain aspects, the methods of measuring IOP include positioning a pressure sensor within the vitreous body such that the entire pressure-sensing membrane of the pressure sensor is maintained within the vitreous body. In one aspect, the IOP measurement of pressure within the vitreous body may be compared to and correlated with a pressure within the anterior chamber, which may be measured according to various other independent measurement methods. This comparison or correlation can determine any degradation or attenuation of the IOP, if any, as it is transmitted from the anterior chamber to the vitreous body. As discussed above, monitoring the anterior chamber directly is not worth the risk of affecting vision significantly or the associated liability. Even if there were a slight degradation or attenuation in IOP when measuring within the vitreous humour, the increased pressure may be detected with a continuous pressure profile that may satisfactorily quantify the relative increase in pressure in the anterior chamber. As one of skill in the art will appreciate, the proposed measurement locations can also be readily validated across a range of animal models, which may also be used to adjust the sensor sensitivity if necessary.

The pressure sensor of the implantable device may comprise a capacitive pressure transducer. In some embodiments, the device includes an absolute reference with a vacuum within the transducer and may include a differential mode using two capacitors for sensing and reference, respectively. It is appreciated however that the first wafer may incorporate other types of sensors or transducers, such as an accelerometer or piezoelectric, depending on the desired physiological signal for measurement and sensing. The capacitive pressure transducer comprises at least a first cavity structure and a second cavity structure, wherein the at least first cavity is distal of the at least second cavity. The at least first cavity is under vacuum so as measure the physiological signal, such as IOP, while the at least a second cavity structure is configured to measure a reference pressure of one more parameters other than the IOP so that it is independent of the actual IOP measured by the at least first cavity. The second cavity has also vacuum but the membrane has a reduced area to significantly reduce the sensitivity to pressure but with the same electrical characteristic (e.g. capacitance).

Since the device is adapted to obtain and store pressure data with relatively low power consumption, in some embodiments ultra-low power consumption (l μW or less), pressure data can be measured and stored continuously for at least a week, preferably several weeks at a time, without re-charging. By holding the external data acquisition/charging device in close proximity to the implanted sensor device, wireless transmission of data and/or charging is initialized and performed rapidly upon detection of the external device, typically in a period of time less than 15 seconds, preferably a few seconds or less. The present methods allow for improved monitoring of pressure of the eye while improving patient compliance by avoiding the complex charging/data transmission routines associated with conventional IOP sensors. In addition, by utilizing an implantable miniature sensor device that transmits measured IOP data and is charged from/within the vitreous body, damage to the surrounding eye tissues can be avoided, which prevents potential patient discomfort and vision damage. Once implanted, the sensor device can provide continuous monitoring up to at least a week, typically several weeks (e.g. two to ten weeks), between charges. The implanted miniature sensor stores the measurements in a memory and may process the data, such as by determining a trend or average, and store the processed data on a memory of the miniature device to be acquired by the external data acquisition/charging device at the next charging. After acquisition from the miniature device, the data may be made accessible to the patient, a treating physician or other health care professional at any time (e.g. by upload to an electronic medical record via a cloud or a central server). The data acquisition/charging device may be incorporated into a personal mobile device as a separate attachment (e.g. ultra-thin casing snapped onto the phone body as a case), such as a smart-phone, tablet, or glasses or other wearable gear (requiring for external transceiver module to offer different form factors) which can be easily held or positioned in close proximity to the eye, such about 2 inches or less, for a duration of time sufficient to transmit stored measurements and charge the miniature device, typically a period of less than 15 seconds, less than 5 seconds, and preferably a few seconds or less in order to be transferred to the user. An audio or visual signal may be sent to the user at the completion of the data/power transfer.

In one aspect, the miniature sensor device is dimensioned sufficiently small to allow delivery of the entire device through a syringe so that the device can be implanted by injection (e.g. through a 19 gauge needle or smaller), which advantageously allows the implantation procedure to be performed in a physician's office without a need for an invasive surgical procedure (also qualified as an in-office minimal surgical procedure). The configuration of the sensor device as well as its placement and stable anchoring within the tissues of the eye, typically the vitreous body, provides increased accuracy and consistency in measuring IOP, for example sampling within 0.25 to 1 mmHg of accuracy. By utilizing ultra-low power consumption in sampling and storing measurement data on a memory of the device, the sensor device allows data acquisition of an IOP profile over a daily cycle (e.g. active, sleep) without requiring patient intervention beyond periodic charging of the device (e.g. every week or every 2-3 weeks). To allow for these ultra-low power features and advantageous aspects described herein, the IOP sensor device may be constructed as a chip-scale device that is hermetically sealed or encased so as to provide stable operation without degradation, drift or failure for years, often upwards of 10 years or so without replacement.

In one aspect, at least a portion of the sensor of the miniature sensor device is a MEMs device formed by a wafer process, and the components associated with sampling, storing, and wireless charging/data transmission are integrated within the miniature device implanted within the tissue in which the measurement are performed. For example, the miniature device may comprises a sensor in which the sensing membrane is hermetically encapsulated and implanted within the tissue in which pressure is being measured (e.g. vitreous body). The one or more coils for wireless charging/data transmission and any associated electrical components (e.g. memory, processor) may be directly coupled with the miniature device or incorporated into the device such that the miniature device comprises a single, integrated device as opposed to discrete components implanted in separate tissues.

### BRIEF DESCRIPTION OF THE DRAWINGS

FIG. 1A is an illustration of an overview of an implanted IOP sensor device wirelessly coupled with an external data acquisition/charging device for communication with a system utilizing an external computer and/or cloud server, in accordance with embodiments of the present invention.

FIG. 1B is an overview illustration of a monitoring treatment system utilizing an implanted IOP sensor device in accordance with embodiments of the invention.

FIG. 2 is a schematic of an implanted sensor device wirelessly coupled with an external device for charging and data transmission, in accordance with embodiments of the invention.

FIG. 3A illustrates a patient charging an implanted IOP sensor device with a portable handheld data acquisition/charging device, in accordance with embodiments of the invention.

FIG. 3B illustrates a schematic of an implanted sensor device wirelessly coupled with an external device for charging and data transmission, in accordance with embodiments of the invention.

FIG. 4 illustrates a schematic of the architecture of an external receiver in accordance with embodiments of the invention.

FIG. 5A illustrates an example embodiment of an implantable sensor device, in accordance with embodiments of the invention.

FIG. 5B illustrates an example interdigitated-coil suitable for charging and/or data transmission in an implantable sensor device, in accordance with embodiments of the invention.

FIG. 6A illustrates a cross-sectional view of the example implantable sensor device in FIG. 5A disposed within a syringe for implantation into a patient tissue by injection, in accordance with embodiments of the invention.

FIG. 6B illustrates a cross sectional side views of the vertically stacked implantable device of FIG. 1A with power receiving and/or data transmission coil.

FIGS. 7A-7C illustrate several views of an alternative design of an implantable sensor device in accordance with embodiments of the invention.

FIGS. 8A-9B illustrate schematics of an implantable sensor device having a reduced width and associated models illustrating displacement of the membranes of the sensor and reference capacitors, in accordance with embodiments of the invention.

FIG. 10 illustrates a schematic of the electrical connections between the battery and the decoupling capacitor in accordance with embodiments of the invention.

FIG. 11 illustrates a block diagram of the process control and power management units of an implantable sensor device, in accordance with embodiments of the invention.

FIG. 12 illustrates a functional block diagram of the ASIC of an implantable sensor device in accordance with embodiments of the invention.

FIG. 13 illustrates a block diagram of the login in a control unit of an example implantable sensor device, in accordance with embodiments of the invention.

FIG. 14 illustrates variations in intraocular pressure potentially undetected by conventional intraocular pressure monitoring techniques.

FIG. 15 illustrates variations in intraocular pressure within a 24-hour period between patient with glaucoma and the normal population.

FIGS. 16-18 illustrate methods of monitoring with an implantable sensor device in accordance with aspects of the invention.

### DETAILED DESCRIPTION OF THE INVENTION

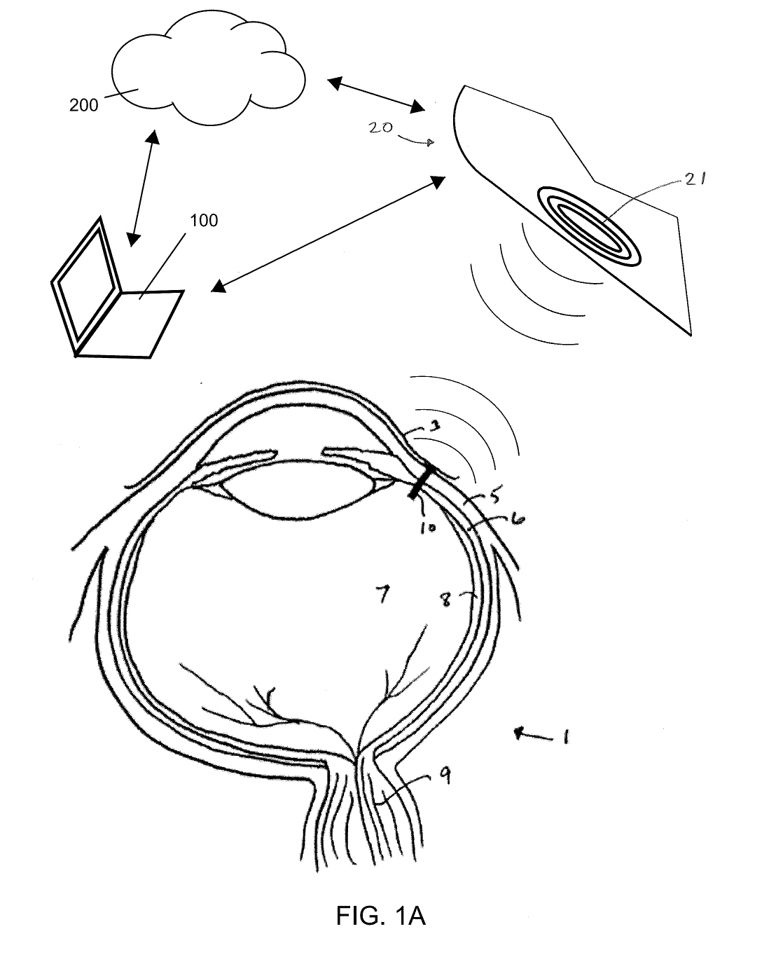

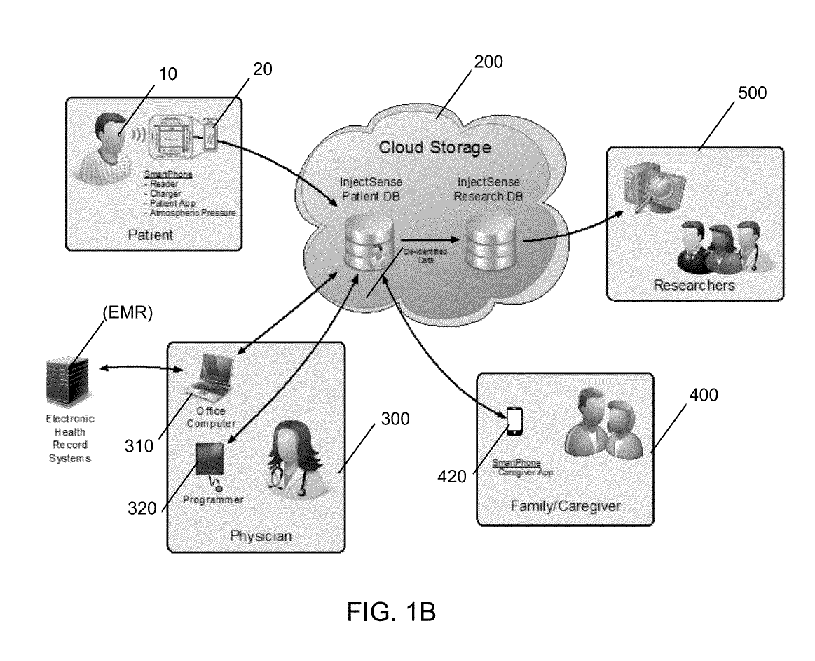

FIG. 1A is an overview illustration of a miniature sensor device 10 implanted within an eye 1 in wireless communication with an external portable device 20 held in close proximity to the eye for charging and/or data transmission. While implanted within the eye, the miniature sensor device 10 obtains multiple pressure measurements over a given time increment according to a sampling program stored on a memory of the sensor device 10 and stores pressure measurement information corresponding to the multiple pressure measurements on the memory of the device powered by energy stored in an energy storage component of the sensor device 10. Upon detection of a coil of an external data acquisition/charging device held in close proximity to the eye having an implanted sensor device, a charging and/or telemetry sequence is initiated in which one or more coils of the sensor device 10 wirelessly couple with one or more corresponding coils 21 of the external device 20 and transmit energy to the sensor device so as to charge the device and transmit pressure measurement information from the sensor device to the external device. In some aspects, the external device may also transmit updates to the programmable instructions stored on the sensor device 10 so as to adjust a sampling program and/or operation of the sensor device 10.

In one aspect, the miniature IOP sensor device 10 is implanted within an eye 1 of a patient by injecting or advancing the IOP sensor device 10 into the eye with a fluid-filled syringe or injector. The IOP sensor device may be positioned within the vitreous body of the eye 1 by penetrating the conjunctiva and sclera with a distal tip of a needle of a syringe 20 along insertion axis I extending through the ora serrata region. Implanting the sensor device by injection at this location is advantageous over conventional implantation methods as it avoids the potential for damaging the delicate structures within the anterior chambers and as well as damage to the photo-sensitive tissues of the retina. In some embodiments, the distal tip of the needle is inserted through the conjunctiva and into the sclera 5 and the choroid 6. The sensor device 10 is then advanced, typically by withdrawl of the needle into the syringe, until at least a distal portion of the sensor device on which the sensor is located is positioned within the vitreous body 7. The sclera 5 is the dense fibrous opaque white outer coat enclosing the eyeball except the part covered by the cornea (not shown), while the choroid 6 is the vascular layer extending between the retina 8 and the sclera 5 to the ciliary body and iris (not shown) of the eye 1. The IOP sensor 10 is disposed within the distal tip 21 of the fluid-filled syringe and may include one or more anchoring members constrained within the distal tip to be deployed upon release (e.g., self-expanding). After release from an injector or fluid-filled syringe, anchoring members at a proximal end of the sensor device 10 may expand laterally outward from the injection axis against the sclera 5 so as to anchor a pressure sensor near the distal portion of the sensor device 10 within the vitreous body 7. By extending the anchoring members along the sclera outside the vitreous body, the anchoring members 17 prevent the sensor device 10 from potentially slipping into the vitreous body, which could cause damage to the retina or optic nerve 9. These implantation methods can be further understood by reference to U.S. Provisional Patent Application Ser. No. 62/019,826, entitled “Methods and Devices for Implantation of IOP Sensors” filed on Jul. 1, 2014, which is incorporated by reference in its entirety. Although the telemetry and charging aspects are described in reference to an IOP sensor, it is understood that these aspects are not so limited and may be applied to various other types of miniature sensors that sense various other parameters and physiological conditions and are suitable for implantation in various other regions of the eye or in other tissues or organs (e.g. vascular, cardiac, cranial, etc.).

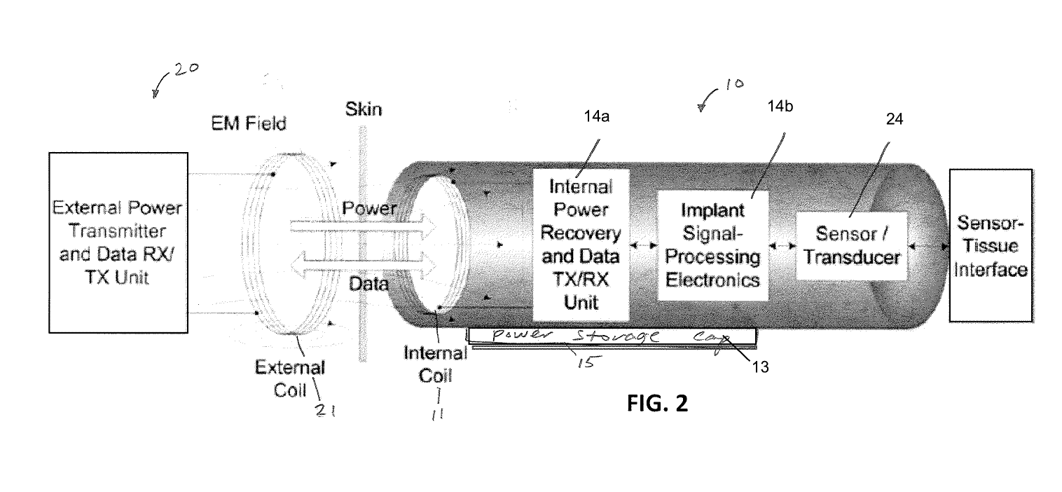

In one aspect, the miniature sensor device is implanted within a targeted location within the body so that a sensing diaphragm (not shown) of the sensing transducer is entirely within the targeted location in which pressure measurements are desired. The sensor device includes a control unit having a processor that controls measurements with the pressure transducer so that pressure can be sampled frequently at multiple times throughout the day at regular intervals, regular intervals being between 5 minutes and two hours, preferably hourly so as to provide a substantially continuous pressure profile over an extended monitoring period. The monitoring period may extend over many months, typically many years, since glaucoma is a chronic condition that must be monitored over a patient's lifetime once diagnosed. In contrast to conventional methods utilizing external means to power sampling or obtain measurements, the implantable sensor device is adapted to store energy within its structure sufficient to power frequent sampling and storage of pressure measurement information over an extended period, such as a time increment of about one week or more, typically 2-3 weeks or more, without intervention by the patient during this extended period of time. To accomplish this, the sensor device 10 includes various control and logic components within the miniature sensor device that manages power consumption of the device so as to provide frequent sampling for substantially continuous monitoring of IOP, some of which are illustrated in the schematic shown in FIG. 2.

FIG. 1B is an overview illustration of a monitoring treatment system utilizing an implanted IOP sensor device in accordance with embodiments of the invention. The sensor device 10 is wirelessly coupled with an external data acquisition unit 20, such as a smartphone, so that physiological measurements obtained by the implanted sensor device are collected periodically by the external data acquisition unit and can be wirelessly communicated to any or all of a physician 300, a family member and/or caregiver 400 and researchers or pharmaceutical entities 500 and may also be used to update the patient's electronic medical record (EMR). The external data acquisition unit 20 may have enhanced functionality in addition to acting as a reader and/or charger and may include a patient application through which the physician can reprogram or update the implanted sensor device 10 (such as through a patient device 320 (e.g. smartphone, tablet) using a physician application. A family or caretaker can also monitor or manage data with a family/caretaker device 420 using a specialized caregiver application. Researchers, in turn, may also utilize similar devices or applications. However, the information sent to the researchers may be de-identified data so as to safeguard the privacy of the patient, while still allowing medical or pharmaceutical research. Typically, the physiological information is stored on a patient database from which the information can be transmitted to the physician or a caretaker, or the data is communicated to a research database from which researchers can use the data for research studies. In some embodiments, the information is stored on a remote server and/or uploaded to the cloud 200

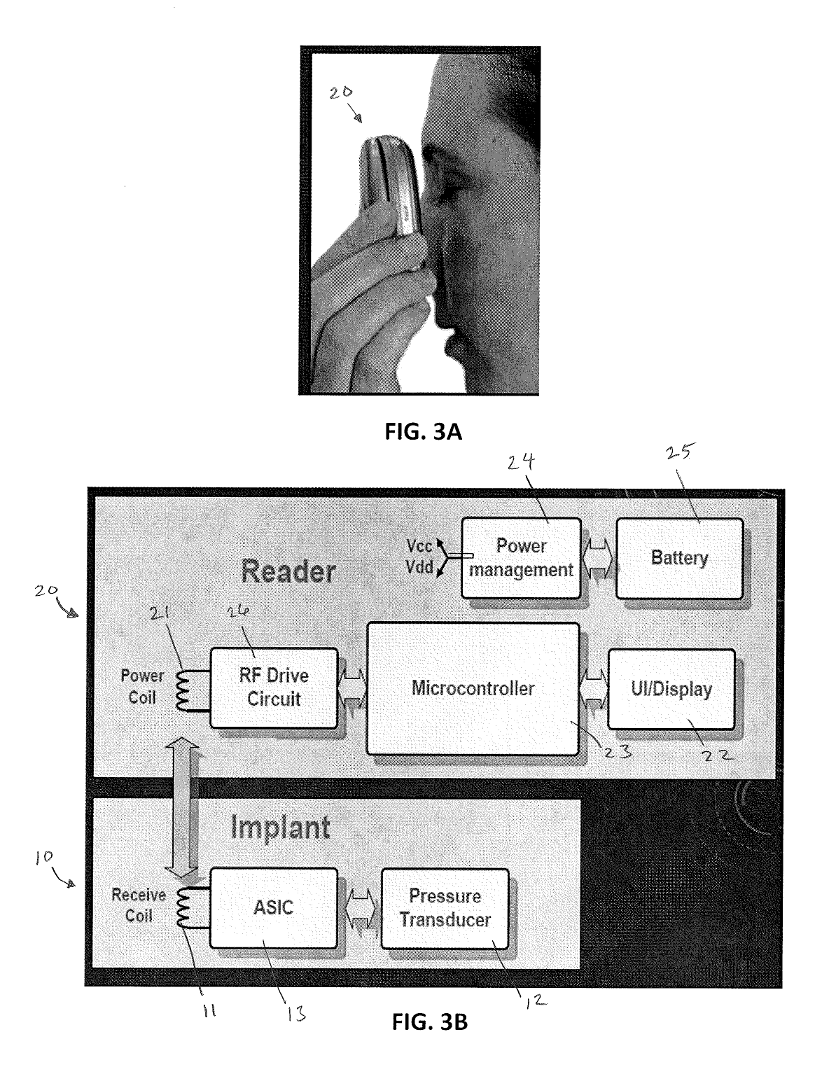

FIG. 2 illustrates a schematic of an external data acquisition/charging device 20 wirelessly coupled with an implanted miniature sensor device 10. The external device 20 includes an external power transmitter unit for charging and a data transmission and receiver unit, which are coupled to one or more external coils 21 for wireless coupling with one or more corresponding internal coils 11 of the implanted miniature sensor device 10 through a skin or tissue of the patient. In addition to the internal coils 11, the implantable miniature sensor device 10 includes an internal power recovery and data transmission and receiver unit 14a, a power storage component 14,15 (e.g. storage capacitor and/or battery), an implant signal processing control unit 14b and a sensor/transducer 12 adapted for measurements of one or more physiological parameters, such as IOP. The external data acquisition/charging device may be configured as a portable handheld device to allow a patient to easily perform recharging and telemetry periodically (e.g. daily with high sampling rate (5 minute) or every week or 2-3 weeks with hourly sampling rate) at their convenience (e.g., at home or work) and without having to visit the doctor or a medical facility. Advantageously, the external device can be incorporated into a personal handheld device, such as a smartphone, which does not require much patient interaction as it can be easily held in close proximity to the eye in which the sensor device is implanted, as shown in FIG. 3A. In this embodiment, the data acquisition/charging device is incorporated into a smartphone, however, it is appreciated that for sensor devices requiring longer durations for telemetry and/or charging, the data acquisition/charging device can be incorporated into a pair of glasses or other wearable device that can be comfortable worn by the patient for the duration required. In some embodiments, close proximity of the smartphone for a short duration of time is sufficient obtain physiological measurement obtained by the sensor device, but charging of the sensor device may require close proximity of a separate charging device for longer durations of time, for example a duration of time greater than 15 minutes, such as a duration of time between about 15 minutes and three hours or between about 20 minutes and 2 hours. In such embodiments, the charging device may be incorporate into a pair of glasses or other wearable device so as to transfer energy to the sensor device through an antennae or charging coils over the duration of time. In some embodiments, the data acquisition unit may be incorporated into the charging device as well. Typically, charging need only be performed about once a week or less, depending on the frequency of sampling.

FIG. 3B illustrates a schematic of an implanted miniature sensor 10 and an external data acquisition device 20 in accordance with certain embodiments of the invention. The implantable miniature sensor 10 includes one or more coils 11 for receiving energy and transmitting/receiving data, attached to a control unit 13 including an application specific integrated circuit (ASIC) for micro electro mechanical systems (MEMS), which is electrically coupled with a miniature pressure transducer 12. The data acquisition/charging device 20 includes one or more coils 21 for transmitting energy and transmitting/receiving data coupled with an RF drive circuit 26 controlled by a microcontroller 23 and may include a user-interface display 22. The user-interface display 22 may be used to view, process or upload the received data relating to pressure measurements to a central server or may be used to configure the sensor device or update programmable instructions of a sampling program stored on a memory of the sensor device 10. Since the external device is typically a portable handheld device, external device 20 may include a battery 25 and a power management unit 24 to control energy discharge from the battery 25 during charging and telemetry sequences.

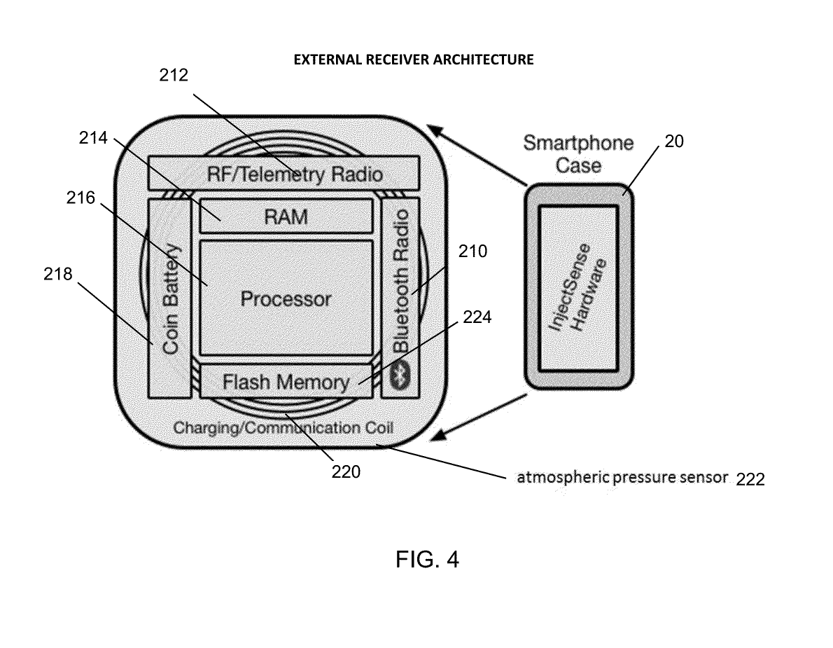

FIG. 4 illustrates a schematic of the architecture of an example external receiver 20, which is incorporated into a smartphone. In this embodiment, the receiver includes an RF/telemetry radio 212, and a Bluetooth radio 210 to facilitate wireless communication; a processor 216 with RAM 214 and flash memory 224 for storing programmable instructions and received data; a coin battery 218 for powering the device; and a charging/communication coil 220 for facilitating charging and/or wireless communication with the implanted device. In some embodiments, the external receiver may also include an atmospheric pressure sensor 222 so as to further refine the physiological pressure measurements obtained by the implanted sensor device 10. The receiver may further include a specialized application for use in performing any of the functions described herein or in switching between or operating various modes (even concurrently). In one aspect, the receiver may be configured to obtain atmospheric pressure sensor data from external sources for correlation with obtained physiological data (e.g. weather data associated with a location of the smartphone based on GPS). It is appreciated that various other functions may be incorporate into the smartphone in addition to those described herein.

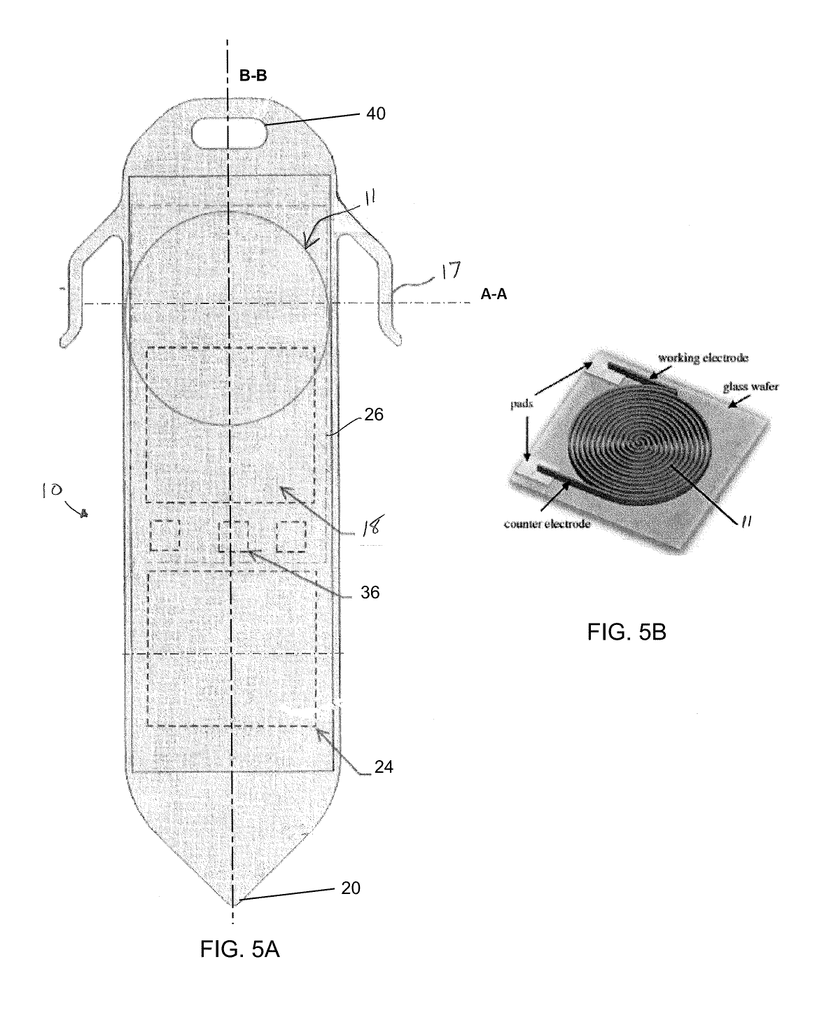

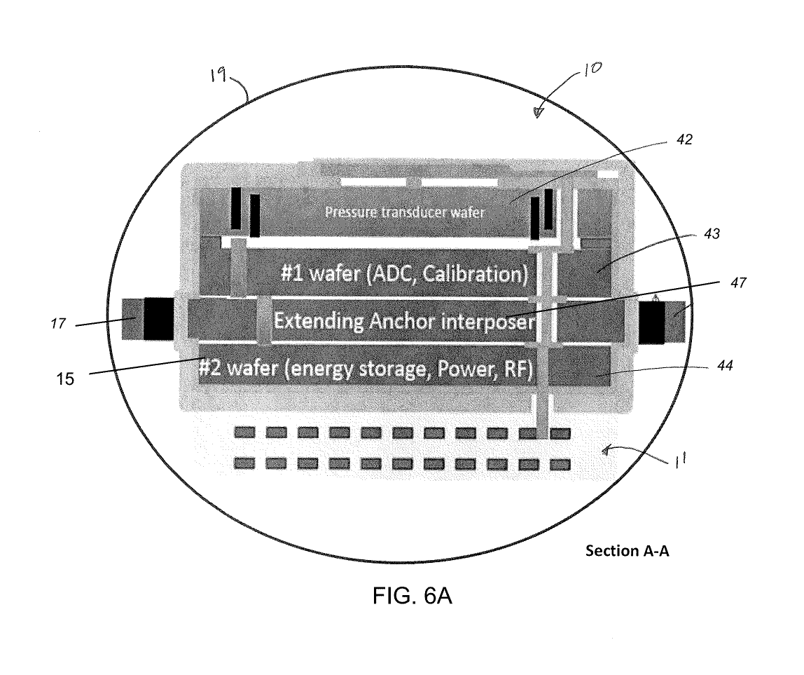

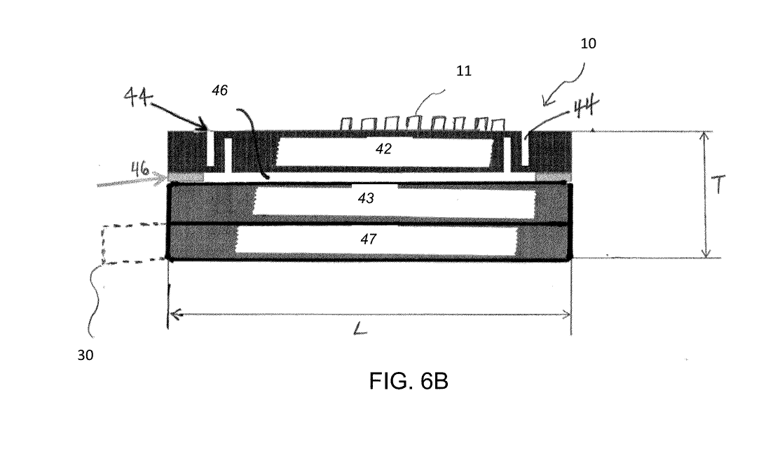

FIGS. 5A-5B and 6A-6B illustrate an example miniature sensor device 10 in accordance with aspect of the present invention. Typically, the sensor and/or antenna feature utilizes MEMs technology such that the entire device can be sized sufficiently small to be implanted within the tissue being measured by the sensor. In one aspect, the miniature sensor device 10 has a length of about 4 mm or less, a width of about 650 microns or less, and a thickness of about 200 microns or less. The sensor device 10 comprises a vertically stacked architecture utilizing one or more wafers, various features being defined in one or more layers of the one or more wafers or attached thereto. Ultra-miniaturization can be achieved with MEMS, IC wafer thinning to dimension below 200 μm thickness, such as thicknesses as small as 50 μm, which allows for implantation of the sensor device 10 into the desired target location by injection. Based on outer dimensions (width and height) to fit within a syringe of gauge 19 (equivalent to 690 μm) which is used as a delivery and protection device. By use of chip-scale integration, the vertical stack can be dimensioned at 600 μm or below with bonded multiple wafers. The vertically stacked architecture and chip-scale of the miniature sensor-device can be further understood by reference to U.S. Provisional Patent Application Ser. No. 62/019,841 entitled “Hermetically Sealed Implant Sensors with Vertical Stacking Architecture” filed on Jul. 1, 2014, the entire contents of which are incorporated herein for all purposes.

As shown in the overview of FIG. 5A, the sensor device includes a pressure sensor 24, which includes a capacitive pressure transducer formed in part by a MEMs device. Typically, the pressure transducer has a full scale range from −100 mmHg to +200 mmHg, compare to 1 Atm (760 mmHg), and more particularly in a range from 660 mmHg to 960 mmHg (absolute), so as to be suitable for use in measurement of IOPs within the human eye. The sensor device 10 may include a MEMs transducer formed in a MEMs wafer near a distal portion of the device to define, at least in part, the pressure sensor 24. Electrical pads 36 may be defined in a more proximal portion to provide a common node connection to electrically connects the MEMs wafer of the pressure sensor to the ASIC wafer of the sensor device (and may also connect an optional reference sensor), though which the pressure sensor 24 is controlled and pressure measurements are obtained.

In one aspect, the sensor device 10 includes one or more anchoring members 17 that displace laterally outward upon implantation so as to anchor the distal sensor 12 within the targeted location for measurement of pressure. The sensor device may also include one or both of a distal penetrating tip feature 30 for implantation and an explanation feature 31 disposed on a proximal end to facilitate explanation or removal of the implanted sensor device 10. The distal penetrating feature 30 may comprise a wedge shaped feature at its distal tip to be positioned at the tip of the syringe or injector, such as shown in FIG. 5A. The wedge shaped features facilitates the insertion of the implant into the eye tissue. The syringe will create the first incision and as the saline solution (also including analgesic solution) within the syringe is pushed, the wedge tip will ease the insertion into its final position through the sclera. Within the syringe, there is no air contact with the impact as it is immerse in saline solution (also including an analgesic solution) and the complete delivery system with implant are sterilized. The implantable sensor device may have a mechanical feature 31 that allows the device to be ex-planted but in passive mode will have no effect on the patient (biocompatible, MRI compatible and not obstructing field of vision). The anchoring mechanical feature may be attached to the implanted device as a separate part that allows the implant device without anchors to be attached to a shunt for glaucoma therapy. Other anchoring features are usable with the implantable miniature sensor device for the monitoring of other physiological parameters such as ICP (cranial pressure), cardiovascular (PAP) and cardiac valve (e.g. as flowmeter) with adapted electronic to address application requirements (e.g. higher sampling rate for cardiac applications at 100 Hz and across a larger gauge pressure of up to 100 mmHg).

In one aspect, the implantable device sensor device uses an ultra-low power circuit technique in a sub-threshold mode (for ultra-low dynamic power consumption and minimized static power consumption or low leakage CMOS process) that allows the device to operate at very low sampling rate autonomously and log the raw data until the external device or base station is wirelessly linked to the device. The data upload is triggered when the base station has re-energized the implanted sensor to be able to operate the embedded RF transceiver within the implant which requires a continuous access to an external power source. After the data upload is completed and implant energy storage (which includes a raw data calibration with stored coefficients before transmission) fully charged; the external unit turns the implant into an autonomous mode (e.g. all internal blocks to the implant are disconnected from power source to reduce leakage (called deep sleep mode) and only the timer (operating at set sampling rate is ON)). If the device is in complete failure mode of operation, there is no impact on the patient since the device is entirely passive, non-radiating, non-irritating or without potential to cause infection. The design described herein allows for an implantable sensor device capable of operating without failure for an extended period of time, typically at least 10 to 15 years, which allows for long term monitoring without requiring periodic surgical procedures or repeated office trips to obtain IOP measurements obtained according to conventional techniques.

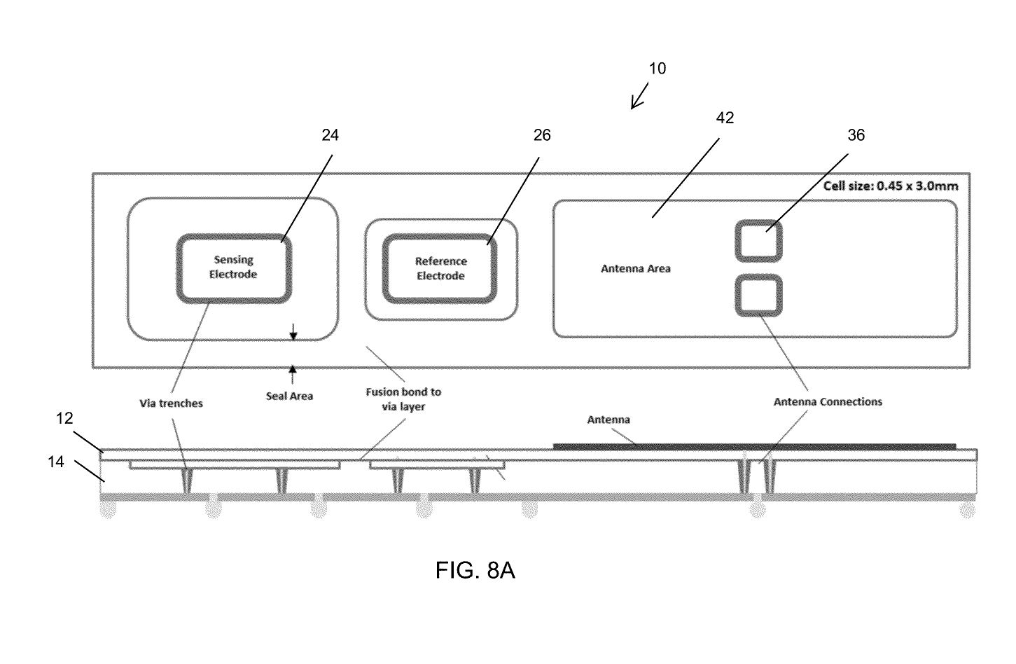

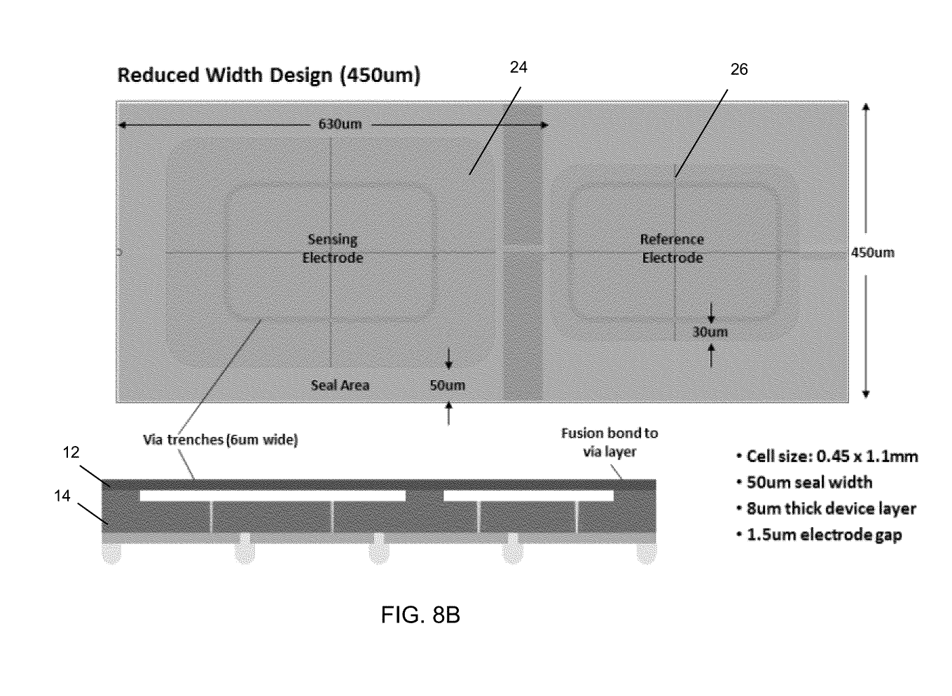

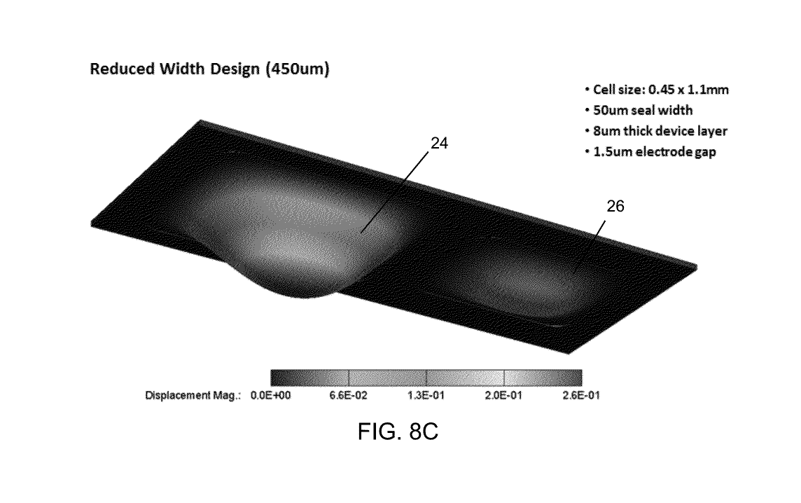

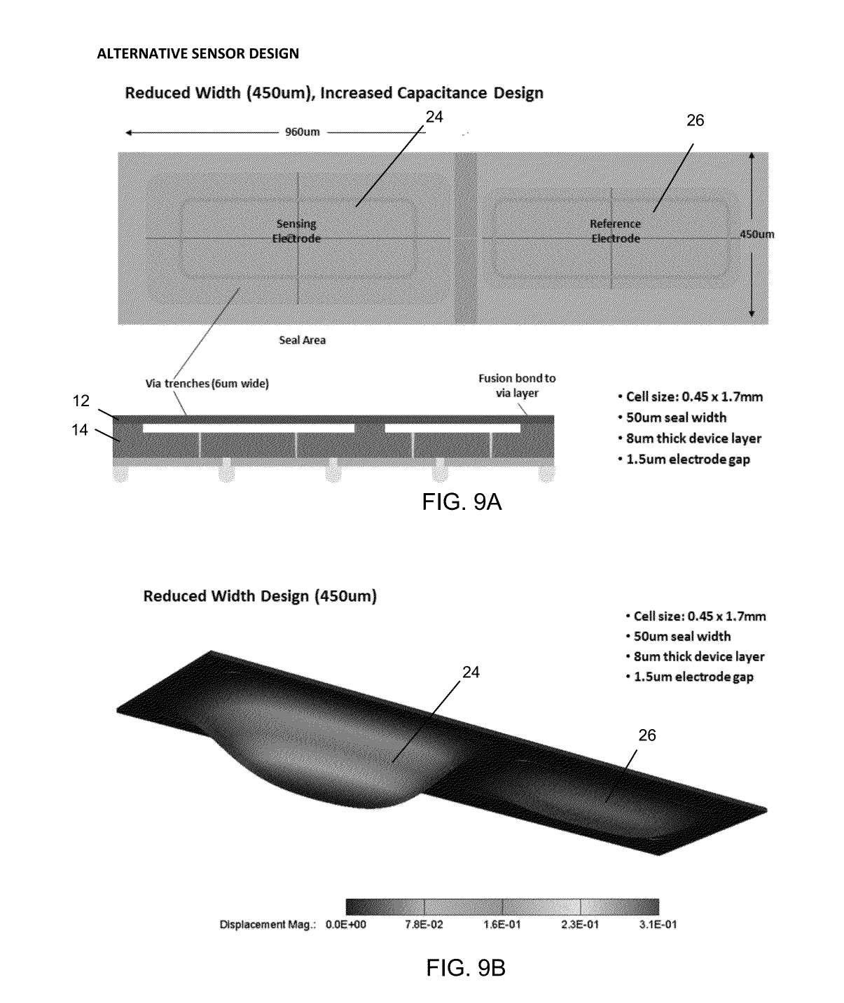

In one aspect, the sensor device may further includes a reference sensor 18, adjacent the pressure sensor 24, which can be used to measure second order effects associated with the pressure measurements obtained by the pressure sensor. The reference sensor 18 may also be formed, at least in part, within the MEMs wafer and may be defined by a substantially similar construction as the sensor device so as to measure second order effects associated with the pressure measurements obtained by the pressure sensor. Variations due to stress in the wafers of the sensor device or changes in temperatures may affect the pressure measurement signal. By including a reference sensor, these variations due to second order effects can be accounted for to improve accuracy of the pressure measurements. In one aspect, the pressure sensor includes a flexible pressure sensing membrane that forms a portion of a sealed chamber under vacuum. In some embodiments, the reference sensor may include a similarly sized chamber and since the reference sensor is not being used to measure pressure, the corresponding chamber is not required to be under vacuum such that the chamber can be filled with oxide) so as to measure a reference pressure of one more parameters other than the IOP (e.g., variations due to stress, temperature, etc.) so that it is independent of the actual IOP measured by the sensing capacitor 24. In other embodiments, both the sensing and reference cavities have a vacuum but are different mechanically. For example, in a reference capacitor 26 which also has a vacuum, in order to remove the sensitivity to pressure, the membrane can be made smaller to increase stiffness but the capacitance is the same for closer matching when used in differential mode (Cₛₑₙₛₑ/C_{ref}). Examples of such configurations having reference electrodes of reduced width are shown in the embodiments of FIGS. 8A, 8B and 9A. It is appreciated that the dimensions shown in the embodiments in FIGS. 8A and 9A are merely examples of device dimensions and should be noted that such devices may be fabricated according to various other dimensions in accordance with embodiments of the invention. For example, any of the dimensions shown may be scaled upwards or downwards (e.g. by 5%, 10%, 20%, etc.) as desired for a particular application.

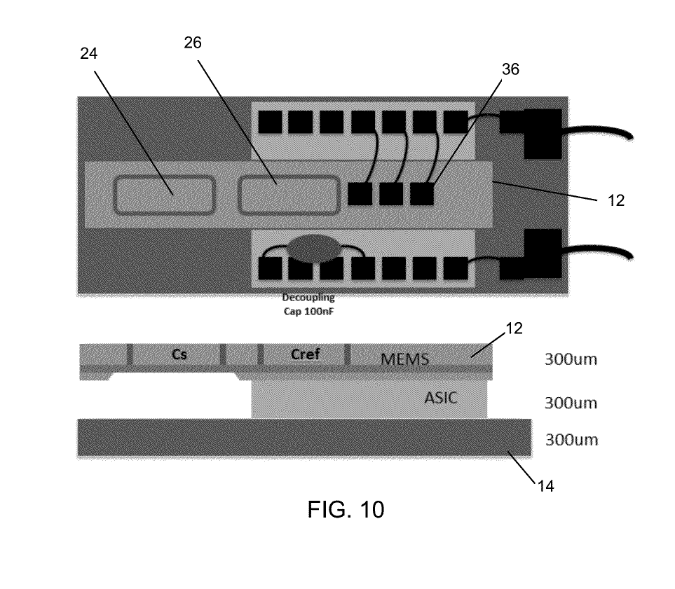

As can be seen in the displacement models in FIGS. 8C and 9B, the membrane of the reference electrode of reduced width has increased stiffness such that its displacement in response to a change in pressure is considerably less than that of the pressure sensor electrode. Advantageously, this configuration makes additional space in the miniature device available for communication/charging coils or various other components as needed. The reference capacitor 26 is positioned within the vicinity of sensing capacitor 24 in order to accurately cancel out noise signals or other artifacts that alter the sensing measurements. Additionally, the reference and/or sensing capacitors 24, 26 may have a post 34 centered therein so as to prevent the top reference and/or sensing membranes 22 from contacting the base structure 28. The pressure transducer will have the sensing capacitor 24 and the reference capacitor 26 with a common node, such as the bulk wafer 12. Typically, the pressure transducer has a full scale range from −100 mmHg to 200 mmHg, compare to 1 Atm (760 mmHg), and more particularly in a range from 660 mmHg to 960 mmHg (absolute). FIG. 10 illustrates a die design schematic showing the electrical connections between the sensor and reference electrodes to the one or more power source/energy storage wafers.

In one aspect, the second order effects can be measured by the reference sensor and embedded in the pressure measurement data transmitted to the external device so that the measurement data can be processed external to the sensor device 10. In another aspect, the sensor device 10 may be configured to process the pressure measurement data and account for the second order effects detected by the reference sensor and store the processed measurement data on the memory of the sensor device 10 for later transmission to the external sensor device. While the various aspects of the sensor device 10 described herein may apply to sensor devices that do not include such a reference sensor, the use of the reference sensor is particularly useful in improving accuracy of pressure measurements obtained with a miniature pressure sensor. While a miniature sized pressure sensor offers various advantages in terms of implantation and where the pressure can be measured, there may be certain challenges associated with such miniature sensors. For example, a pressure sensing membrane of a miniature or ultra-miniature sensor device is considerably smaller than many conventional pressure sensing transducers such that accuracy may be reduced. For example, various factors (e.g. changes in temperature or stresses within the device) may affect the pressure measurement signal in a miniature pressure sensor to a greater degree than would occur in a substantially larger membrane. Therefore, by including a reference sensor of substantially similar construction adjacent the miniature pressure sensor, these second order effects can be measured and accounted for, thereby allowing the miniature pressure sensor to obtain pressure measurements of accuracy approaching or even exceeding those of substantially large pressure sensors.

In another aspect, the sensor device 10 includes an energy storage component 15 that stores sufficient energy to obtain multiple pressure measurements each day for a time increment of at least one week, preferably hourly measurements for two-three weeks. Typically, the energy storage component 15 includes an energy storage capacitor formed, at least in part, by a wafer on a backside of the sensor device opposite the pressure sensor 24 and reference sensor 26. In other embodiments, the energy storage component may include a rechargeable battery. Since the reference sensor 15 does not measure pressure, the energy storage capacitor can be positioned to overlap with the reference sensor 15 so as to maximize the size of the energy storage capacitor on the miniature sensor device, which allows sufficient power to obtain and store pressure measurements for at least one week, often two or three weeks or more.

In embodiments having a reference sensor, such as described above, the one or more coils can overlay the reference sensor so as to maximize the size of the coils for the miniature sensor device to allow for substantially rapid charging and transmission/receiving of wireless communication through the one or more coils 11. In one aspect, the one or more coils may include dual-stacked coils, one coil being adapted for receiving energy through inductive coil to charge the energy storage component, while the other coil is adapted for wireless communication to transmit or receive data associated with pressure measurement sampling. The implanted device provides interface to the media and connected to an antenna for power and data transfer and its sidewall are coated with Ti (for a second hermetic barrier) and PPMA to provide soft contact to tissue in particular to round device edges which may be functionalized with anti-inflammatory solution to minimize irritation/immune system response. In one aspect, the coils for inductive coupling may be located outside of the hermetic barrier of the implant. The coils may be defined with dielectric layer and coil layer which are typically in the range of 20 μm to 30 μm thick for coils made of Au or other biocompatible material. In some embodiments, two coils are used separately one for power and the other for data. In the present example, power and data transfer can be operated in separate phases such that subdivision of coils is not required. In some embodiments, the one or more coils may be defined as a 3D interdigitated double-coil in which two coils that are coiled within the same layer, such as shown in FIG. 5B. The interdigitated is shown for illustrative purposes on a glass wafer substrate. It is appreciated that such a coil could be attached to various other types of substrates such as a silicon bulk wafer of the sensor device shown in FIG. 5A.

FIG. 6A illustrates a cross-sectional view of the sensor device 10 shown in FIG. 5A as it would appear disposed within a needle 19 of a liquid filled-syringe before deployment. As can be seen, the laterally extending anchors 17 are defined within an interposer layer 47, which may comprise a silicon wafer, the laterally extending anchors 17 constrained inward within the needle. The pressure sensors, reference sensors, and various logic components that control sampling with the sensor, power management and charging and telemetry may be included within one or more other layers and wafers within the vertically stacked construction of the miniature sensor device. For example, the anchoring members may be defined within an interposer wafer, the ADC and calibration features can be defined within a bulk wafer 43 disposed on top of the interpose wafer 47, the pressure sensor 24 can be formed, in part, by a pressure transducer wafer 42 attached to wafer 43. The energy storage capacitor, as well as the power management and telemetry logic can be included in wafer 44 attached to the underside of the device, to which the one or more coils 11 utilized for charging and telemetry are also attached. This vertically-stacked construction is but one example of a miniature sensor device that may be realized in accordance with the aspects described herein. It is understood that various other configurations and constructions of miniature sensors may be used in accordance with the principles and methods described herein.

FIG. 6B illustrates a cross-sectional view lengthwise of an embodiment of the implantable sensor device 10. In this embodiment, the MEMS wafer 42 is vertically stacked or disposed over a CMOS wafer 43 so as to form a first hermetic seal. In particular, the vertical stacking of the wafers is configured to create a hermetically sealed cavity 46 between the MEMS wafer 42 and CMOS wafers 43 of the implantable device 10.

This approach of wafer or die stacking is sometimes referred to as “chipscale packaging” within the electronics manufacturing field. Chipscale packaging is well understood by those of skill in the art in the MEMS/CMOS manufacturing industry, and is of particular benefit to the present invention in enabling production of smaller, integrated wafer assemblies that are easier to manufacture, provide improved performance, and are less expensive. In particular, constructing the implantable device 10 based on this vertical stacking approach allows for the implant form factor (e.g., dimension, size, shape, volume, etc.) to be significantly reduced (e.g., by a factor of 10×). Conventional implants typically require titanium, ceramic, glass or like outer packaging, which adds to the overall size and bulkiness of such conventional implants. The present invention advantageously employs vertical stacking to define its own hermetic package, which encapsulates all the electronics. As such, the implant 10 architecture and resulting form factors allow it to be easily implanted as an injectable and within a desired location within the eye of a patient.

In particular, at least one coil 11 is illustrated for wireless charging of the battery-less implant and data communication with an external base station (e.g., glasses, phone, etc.). In this figure, the least one coil 11 is vertically stacked or disposed over the first wafer 42 and the reference capacitor 26 while the distally positioned sensing capacitor 24 (see FIG. 5A) remains exposed and entirely disposed within the vitreous body for accurate and direct IOP measurements. The coil 11 may be defined in terms of topology to provide the highest inductance, which is dependent on the depth of implantation and energy transfer efficiency. The first phase of operation may be recharging of the implant 10 while the second phase may be data transfer to recover and record logged data. An overview schematic of the example implantable device 10 of FIG. 1 is shown in FIG. 5A, which depicts the locations of the coil 11, reference capacitor 26 and sensing capacitor 24 on the device. It is appreciated that various other configurations may be used in accordance with the aspects of the present invention described herein.

As described above, vertical stacking of the implant 10 is configured to create a hermetically sealed cavity 46 between the MEMS and ASIC wafers 42,43. For example, a gold sealing ring 46 or flange may be disposed between the first and second wafers to create this first hermetic seal between the MEMS 42 wafer and ASIC 43 wafer. The implant may further incorporate a second hermetic seal by depositing a dielectric layer, such as silicon dioxide, over the implantable device and a titanium barrier over the deposited dielectric layer for a third hermetic barrier. This redundant hermetic sealing ensures chronic implantation and provides enhanced sensing stability. Still further, a biocompatible polymer coating, such as parylene, polymethyl methacrylate (PMMA), and like polymers, may be disposed over the titanium barrier to minimize any immune system response (e.g., rejection of implant).

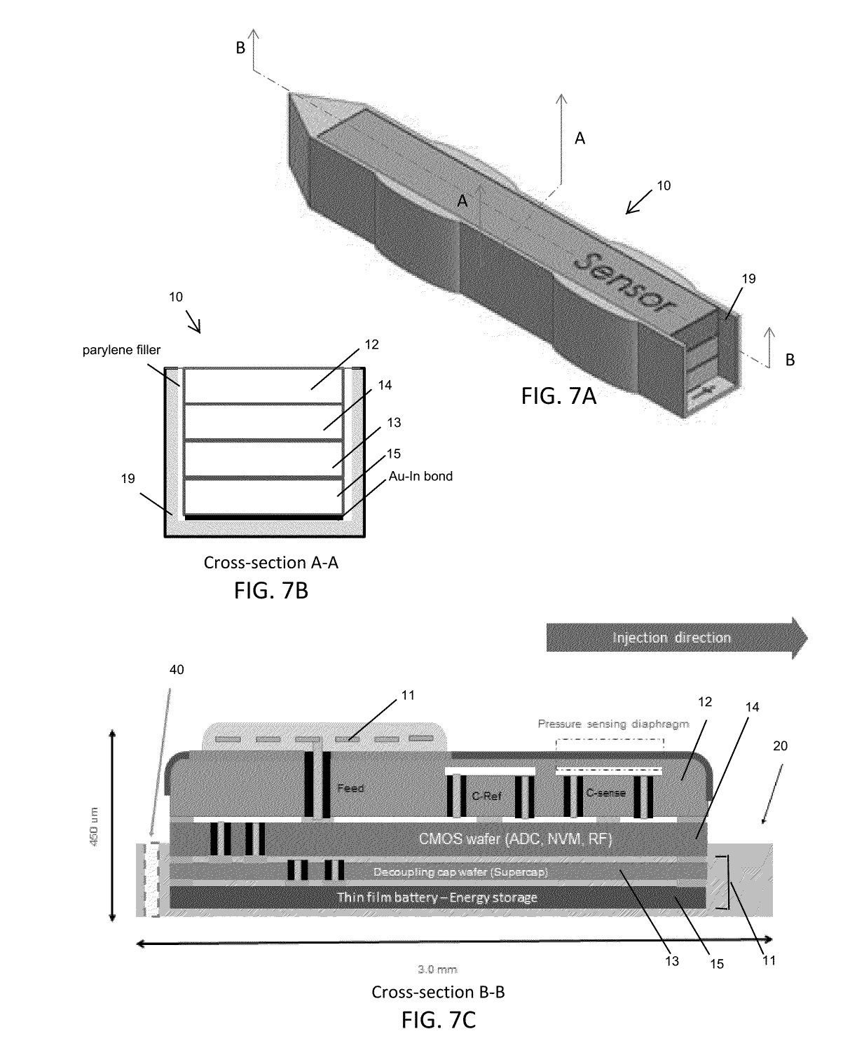

In some embodiments, the stack includes one or more additional wafers, for example one or more wafers adapted for use as a power source. Such embodiments may include a third wafer that includes a supercapacitor. In some embodiments, the stack further includes a fourth wafer that includes a battery. Such embodiments may utilize a power management scheme switching between the supercapacitor and battery in order to perform impedance conversion and provide more efficient power discharge from a high impedance thin-film battery, such as a LiPON battery. Use of the battery to directly power the sensor device is not feasible due to drawbacks associated with high impedance. Electrical impedance is the measure of opposition that a circuit presents to a current when a voltage is applied. High impedance refers to the point at which a circuit allows a relatively small amount of current through, per unit of applied voltage. High impedance generally refers to an ohmic value of about 30K Ohms range. In comparison, a typically power supply is generally about only a few Ohms. An example of such a configuration utilizing a high impedance battery is shown in the embodiment in FIGS. 7A-7C. As can be seen in the cross-sections A-A and B-B in FIGS. 7B and 7C, respectively, the stacked sensor device of FIG. 7A includes the MEMS 12 and CMOS wafers 14, a decoupling capacitor wafer 13 and a thin film battery/energy storage wafer 15. In one aspect, the wafers of the stack may be bonded together with low temperature Gold-Indium (Au—In) bond, while the cavities are formed using a silicon-to-silicon fusion bond. This configuration provides improved thermal budget management, while the silicon-to-silicon fusion bond provides long term vacuum stability (e.g. greater than 20 years). In this embodiment, rather than an interposer layer 18, the stacked device is placed within a support structure or boat 19. An example of such a “boat” can be seen in the embodiment of FIG. 7A.

In some embodiments, an anchoring structure is formed in a separate support structure or the “boat” in which the diced multi-wafer stack is placed and attached with low temperature metal alloy. In some embodiments, this support structure or boat may also include a distally tapered tip 20 to facilitate penetration through the sclera during implantation and may also include one or more anchoring features 38. Such features may be included as components with a mechanical function that clamps onto the sclera (e.g. a proximal and distal anchor on opposite sides of the sclera). The anchoring feature may also include an anchoring loop or extensions. Such anchoring features may be formed of Silicon, Titanium, shape memory alloy, or other suitable materials. In some embodiments, the boat is formed of a monolithic material and include side-walls that extend upwards, at least partly, along a thickness dimension of the stacked sensor device 10.

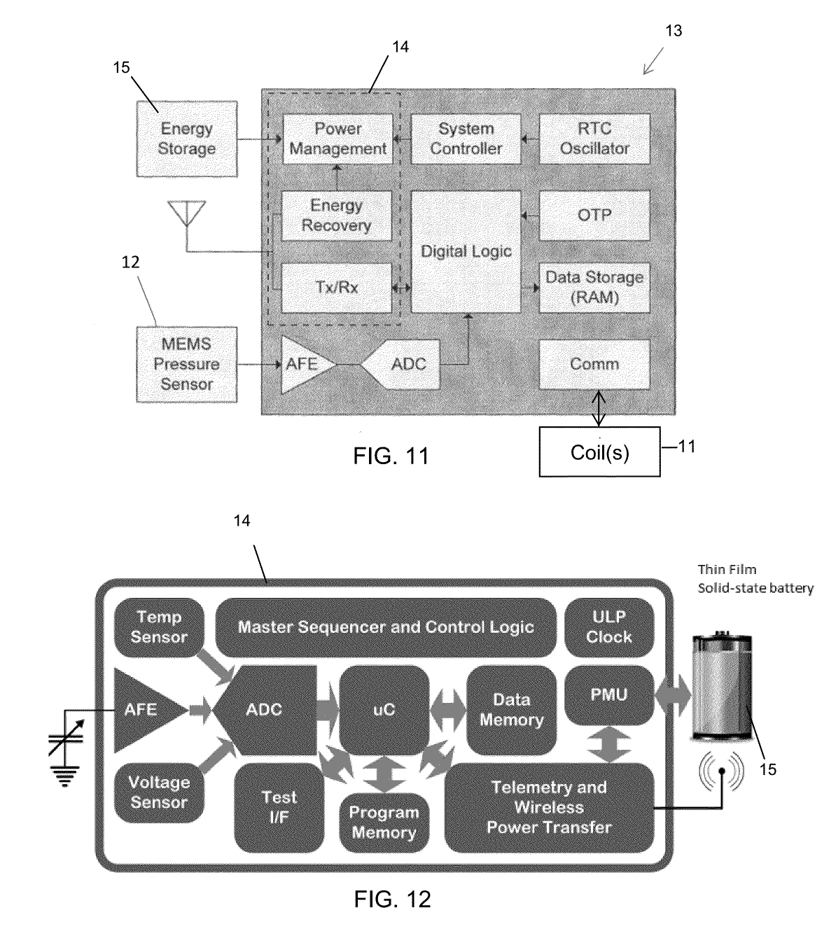

The ASIC wafer 43 may further comprise a radio frequency link, power storage, and/or data storage so as to maximize the wafer topology along its length and reduce the manufacturing complexity and costs of the stacked implant 10. FIGS. 11-12 illustrate example ASIC block diagrams illustrating the various functions of the ASIC wafer 43, such as signal processing, ADC, energy/power management, data acquisition and logging, radio frequency link, calibration, etc. The implantable device 10 may be entirely formed from the same substrate material, preferably silicon wafers or dies and have rounded or anti-traumatic edges to minimize any collateral tissue damage during positioning or implantation. The approach of using silicon material throughout the wafer stack (MEMS 42, ASIC 43, interposer layer 47) offers temperature coefficient of expansion (TCE) matching which enables the mechanical stability of the overall implant 10 and reduces measurement drift. The optional distal penetrating feature 30 may be formed in the interposer layer 47 in which the anchoring features 17 are formed. The pressure transducer 24 may also be embedded with mechanical stress isolation features 44 to decouple any intrinsic stress associated with the vertical stacking architecture, and in particular the TSV electrical connections and/or sealing ring 46. In particular, at least one stress isolation feature 44 may be incorporated into the MEMS wafer 42 to mechanically decouple the pressure sensor from the ASIC wafer 43.

One of the unique features of the miniature sensor device 10 described herein, is the chip-scale packaging approach that allows reduction of implant dimensions by a factor of 10. In typical implanted devices, a Ti, ceramic or glass outer package is required for chronic implant. In the case of a chip-scale packed approach, the device defines its own hermetic package which is encapsulating all the electronics for long term implant. The materials used for bonding are required to be biocompatible such a gold, Ti, etc. In one aspect, use of organic materials within the device is avoided so as to provide increased stability and avoid outgassing or creep of material. The outside surface of the injectable device may be coated with a polymer for soft contact and rounding of the edge. The injectable device may include an anchor structure folded within the syringe that allows long term storage and protection. The exposure to saline within the syringe does not degrade operation of the device. The device can be tested after assembly within the syringe without requiring to break the sterilize barrier or pouch. Prior to injection of the device, a final test and readout of the unique ID can be completed from any external device or base station adapted for communication with the subject sensor device. These aspects allow for a sensor device that is robust enough to provide long-term monitoring, for example over a period of 10 years or more, yet small enough to be injectable as a single device into the tissue. While this approach provide many advantages, it does present certain challenges associated with accuracy of pressure measurements obtained by an ultra-miniature MEMs type sensor, charging of the device, power management and telemetry, which are addressed by the methods detailed herein.

In another aspect, the device can be calibrated through a precision chamber and with multiple units in parallel for cost effectiveness. For example, a batch of 10, 25 or 50 units can be calibrated at the same time. The sensor device may be calibrated by obtaining measurements with the sensor in a controlled environment in one which one or more parameters (e.g. pressure, temperature) are controlled. Measurements are obtained at differing values of the one or more parameters (e.g. high and low temperature and pressure) so as to determine variations in measurements associated with the mechanics of each particular device. The variations may be quantified in terms of calibration coefficients. The calibration data for each sensor device may be stored on a memory of the device for use in processing of data obtained with the device by the sensor device or after transmission of the measurement data to the external device.

A chip-scale approach of integrating a capacitive pressure transducer with its own digitizing IC provides hermetic encapsulation of the electronic for in-vivo implant and with embedded stress isolation, provides measurement stability (e.g. low drift) over time. Stacking the MEMS pressure transducer with back side contact using silicon through wafer via provides an interface to the media being measured (e.g. anterior chamber, vitreous body, or cranial chamber) with a single electrode which minimizes parasitic capacitors such as noise coupling. The approach using silicon material throughout the wafer stack provides temperature coefficient of expansion matching which enables the mechanical stability of the overall device and reduces measurement drift for a chronic implant. The overall integration of digitization IC and telemetry interfaces is implemented using multiple die stacks that are connected through wafer VIA, which requires only low area density and within hermetic encapsulation. The telemetry sequence may be configured to acquire data from the measurement sensor and store measurement information (e.g. raw or processed) on a memory, such as electrically erasable programmable read-only member (EEPROM) to be stored until subsequent charging or a telemetry event. One or more coils may be configured as antennas to wirelessly transmit the stored measurement information. The antenna can be included on the back side of the ASIC and connected through silicon via to a RF power amplifier.

In some embodiments, the sensor device comprises a sensor (e.g. digitized capacitive transducer) with embedded energy storage such as a storage capacitor to allow sampling at a rate in the order of at least 1 sample/hour (duration 1 week) or at higher sampling rate of 1 sample/5 minutes for one day, with ultra-low power operations, such as operating at about 1 μW of power or less. In some embodiments, the embedded energy storage allows for operation of the device without a battery, while in other embodiments the embedded energy storage may be used in conjunction with a battery, such as a thin-film battery (e.g. LiPON), with an advanced power management system. Often, a thin-film battery has relatively high impedance such that power discharge from such a battery can present certain challenges. By switching between the embedded energy storage (e.g. capacitor or supercapacitor) and the battery, energy discharge from the battery is stored in the embedded energy storage and used to power the device. This rapid switching between the battery and the embedded energy storage is managed by a microcontroller. This approach allows use of a high impedance battery while avoiding the challenges associated with using a high impedance power source.

In some embodiments, the sensor device may be charged by recovering magnetic energy through a coupled coil which is fed through a voltage rectifier to power the implant for data acquisition and logging for a targeted period of one week with a total of at least 148 samples. The data measured by the pressure transducer (absolute pressure) is calibrated in pressure and temperature with minimal computation requirements and all calibration coefficients are stored onboard the implant. This data logging may be done autonomously and stored within an EEPROM memory which is capable of storing the information until the external device or a base station is linked to the implant. If the device is out of power, the data may remain stored in memory for up to 10 years without loss of data. The measurement data logged may be collected through the wireless interface with the external device or Base station providing power and data interface during the data transfer. The Base station or external device is able to read the unique identifier of the implant stored in EEPROM and encrypt the transmitted data. In one aspect, the wireless interface uses a modulation scheme that allows for a low data rate similar to RFID (13.57 Mhz or higher to reduce the antenna size) or any comparable scheme. The wireless communication mode can be configured to only operate when the external device or Base station is detected in close proximity. The implant device configuration may depend on several factors, such as the wafers used, technique of stacking wafers having ultra-thin profile, and/or wafer thinning techniques, which may be used to define features that provide additional functionality. For example, features within the miniature implantable device may include power management and post data processing implemented within a form factor that for an injectable device. The implanted device may also be scaled to larger sizes for monitoring context where the depth of the implant is greater than 2 inches such as cardiac applications (e.g. pulmonary artery or cranial applications).

FIG. 11 illustrates a block diagram of the application specific integrated circuit (ASIC) interface with the micro electro mechanical systems (MEMS), which may be used in a control unit of the pressure sensor device. The control unit of the sensor device may include various feature and functions including: incorporating analog to digital converter (10 to 12 Bits), calibration (3^{rd} order) and data logging (168 samples for 1 week at 1 sample/hour), power management that allows for ultra-low power sample acquisition, linearization and wireless data transfer (e.g. less than 1 μW), high power supply rejection ration, various other advanced power management features. The ASIC may be configured with a unique ID, such as an RFID, that can be readily detected and associated with the physiological measurement data measured by the device during transmission of the data to an external data acquisition device. The control unit may be include an RF modulation scheme that allows for ultralow RF power requirement with short range data transfer and/or post processing of antenna to adapt to target transmission/distance. These aspects facilitate wireless charging by holding the external charging device within close proximity to the eye, such as 12 inches or less, for a relatively short time duration, such as 10 seconds or less.

In one aspect, the miniaturized sensor device includes one or more coils for charging of an energy storage component of the sensor device. In embodiments utilizing two or more coils, the coils may be stacked (such as shown in FIG. 6A or may be configured as an inter-digitated coil, such as shown in FIG. 5B. Such sensor devices may be charged via magnetic coupling between a coil of the sensor device and a corresponding coil of the external data acquisition/charging device. A current going through the external coil induces a voltage on the receiving coil of the sensor device. This voltage then goes through a voltage regulator and rectifier that provide a stable power supply to the implant. A decoupling capacitor may be incorporated inside the sensor device to provide energy storage that lasts for at least one week of use for continuous monitoring at frequent sampling (e.g. at least daily, typically multiple times per day or hourly). Regarding wireless coupling between the coils, the ideal case is when both coils are coaxial (aligned on the same axis), since misalignment between coils reduces the transfer efficiency which can go down very quickly. In this case, if the coils are misaligned, it may take longer to recharge the implant, but since the power requirements for charging and operation are very low, typically less than 10 μW and preferably about 1 μW or less, the fully charged state can still be reached within a relatively short period of time, such as less than 30 seconds or, 10 seconds or less, or preferably three seconds or less.

In another aspect, in regard to charging the device, the sensor device may be configured to charge the energy storage capacitor by magnetic coupling with rectifier/regulator or in some case electro-magnetic wave propagation coupled with Cockcroft-Walton rectifier. This allows for improved power transfer efficiency depending on the depth of the implant at the optimum frequency. One factor taken into account is the specific absorption rate requirement (e.g. heating value of RF energy radiated on human body). Utilizing coupled coils allows power transfer and data transmission within these requirements. In some embodiments, a power link separate from the data link (e.g., dual antenna/coil) may be used.

In regard to sampling, in one aspect, the sensor device is configured to obtain sampling every hour for at least one week (24/7). In another aspect, the sampling rate may be adjustable to sampling at every 2 hours or every half hour. In general, the sampling device utilizes very slow sampling at around 12 bits resolution. When sampling IOP, sampling at higher rates is generally not required due to the slow behavior of IOP, such that substantially continuous monitoring can be accomplished by sampling every hour. The basic principle is to sample the IOP at the lowest rate (Nyquist) possible but still represent the signal accurately. When sampling in other applications, however, such as cardiovascular and cranial monitoring, higher sampling rates may be required. For example in cardiac monitoring, higher sampling rates, such as 250 S/s may be desired.

In regard to power management, in one aspect, the control unit manages discharge of energy from the power storage component, typically including one or more storage capacitors (e.g. multi-layer capacitors) to allow the sensor device to obtain a particular set of samples (e.g. 24 samples per day collected hourly) or to collect samples at a particular sampling rate (e.g. hourly or variable based on one or more measured physiological conditions). As the plurality of pressure measurements are obtained, the measurements are stored in a memory of the miniature implantable device, such as EEPROM. A unique ID associated with the sensor device is also stored on the memory such that any pressure measurement information acquired from the memory can be associated with the device. This allows the data to be compiled and processed in a central location (e.g. central server accessible by medical practitioner) even if the information is acquired from different data acquisition devices. The external device may be configured to data log pressure measurements acquired from the sensor device for download to a base station or external device.

In one aspect, the ASIC interface controls power consumption of the miniature sensor device to allow for the advantageous features noted above. Typically, the sensor device includes an energy storage capacitor that stores sufficient energy to operate the sensor device for at least a week, preferably several weeks. The power management circuits and controllers described above regulate drain of power from the energy storage component over the duration of monitoring, typically at least a week. In some embodiments, the device may operate directly from a battery supply, of which there is a wide-supply range, to eliminate the need for any regulation. Utilizing one or more energy storage capacitors rather than a battery is often preferred, however, as this allows for energy storage within an implanted device while avoiding the presence of chemicals commonly associated with batteries within an implanted device. In one aspect, the power consumption budget is divided between the number of samples desired for a single charge, for example hourly sampling for one week for 168 total samples. Different techniques on the architecture/circuit-level may be applied to minimize on-time (burst) current consumption, such as any of the following aspects: low-power front-end and ADS, fast power-up to settling time, smart power sequencing (dynamic power management), high efficiency DC-DC converter, sleep mode at ultra-low static or leakage current. While various aspects of operation may be minimized or suspended to conserve power, certain sub-blocks of operation may be configured to remain activated during operation (e.g. timer providing the heartbeat of the implant), for example, the RTC oscillator, power management, system controller, or any various other aspects. In one aspect, the sensor device may utilize various ultra-low power features (power-management, on-chip oscillators) for autonomous operation (silicon-verified), such as any of those developed by the ASIC design partner.

Data acquisition may be performed by RF energy transmission. The sensor device may be configured with a 2.4 GHz RFID FSK transmitter with an RF energy detector (e.g. EM MARIN 0.18-um/1.8V technology, lower-power RF energy detector for transmitter wake-up in RFID applications) or other suitable RF transmission/detection components. In one aspect, this feature of the sensor device may be configured to operate in a similar fashion as in a passive RFID chip, in which the RF energy transmission from an external reader device powers data transmission from the sensor device. This aspect is advantageous as it allows data transmission from the device to be powered without depleting the reserves of energy stored within the sensor device. In embodiments having a single coil, charging and data transmission would generally be performed according to a particular sequence in which the single coil is used for each function, while in embodiments having multiple coils, charging and data transmission may be performed using a particular coil dedicated for each task, either concurrently or according to a particular sequence. Data is typically stored on a memory of the sensor device, such as EEPROM, that the saved measurement data information can be stored without consuming substantial power and can be stored on a memory of the sensor device for an extended period of time, at least the desired time increment, preferably for many weeks or months if needed.

In one aspect, if the sensor device is not re-charged or the data acquired by the end of the desired monitoring duration, the sensor device may operate in an auxiliary sampling mode configured to further reduce power consumption and/or sampling frequency. For example, to avoid a lapse in monitoring data, in the auxiliary mode, the sensor device may use any remaining stored energy to sample at a reduced frequency, such as sampling every two or three hours or sampling at less than ten times at regularly spaced intervals. In addition, this aspect may include additional auxiliary modes, each having increasingly reduced power consumption, such that when the sensor device is eventually re-charged and stored data acquired, a lapse in measured data can be avoided. This aspect is advantageous if for whatever reason (e.g. loss of the external device or smart-phone, malfunction of external device), the implanted sensor device is not recharged or the stored measurement data is not acquired within the anticipated time increment.

In another aspect, the external device may be integrated within a personal handheld device, such as a smart phone, such as through an application downloaded to the device and/or through additional hardware connected to the device. When integrated within a personal handheld device, the external device may be configured such that normal usage of the handheld device or smart-phone is sufficient to allow charging and transmission of measurement data from the device. For example, an application of the external device may track the time of last charging/data transmission and when sufficient time has elapsed (e.g. 6 days or more), wireless communication with the sensor device is initiated when the personal handheld device is in use and charging and data transmission, such as by RF energy transmission and/or inductive coupling, is performed without initiation by the patient. This aspect allows for improved performance and monitoring as it does not require the patient to perform any particular tasks associated with charge/data acquisition apart from normal every-day use of the personal handheld device.

FIG. 12 illustrates a block diagram of another application specific integrated circuit (ASIC) for use in a control unit of a sensor device in accordance with embodiments of the invention. Notable aspect of a sensor device system for which the depicted ASIC of the depicted ASIC is utilized include use of an absolute pressure sensor, a temperature sensor, a microcontroller, embedded memory, a master sequencer and ultra low power clock generator, power management unit, telemetry and wireless power transfer and a testing interface. In some embodiments, the sensor device includes an absolute pressure sensor of suitable resolution and accuracy for a given application. For use of such a device for measurement of IP, the absolute pressure sensor may have a 0.15 mmHg resolution and an 0.5 mmHg accuracy within the range of 520-860 mmHg at 11 bits, a temperature sensor having 16 mV accuracy within the range of 0-4.1V at 8 bits, and an 8-bit microcontroller. The embedded memory may include a Program memory for storing programs, device ID, trim coefficients and use mode flags (e.g. ULP NVM memory) and Data memory for use in storing pressure, temperature and voltage values, for example ULP NVM memory at 256×3B to allow support of autonomous operation of 1 week at 1 sample per hour rate and 1 day at 1 sample per 5 minutes rate. The master sequencer and ultra low power clock generator may utilize a sleep-wakeup control based on the clock generator (e.g. the ULP clock). The power management unit may include includes reference voltage generator, on-board regulators and battery charger. The telemetry and wireless power transfer may be configured to operate in the 2.4 GHz ISM band, downlink for data, uplink for implant configuration and allow 50 μW power transfer capability from 3 cm, capable of charging the battery in a couple hours or less, preferably about 30 minutes or less. The system may further include a test interface that allows for production testing and programming by the user.

In one aspect, the sensor device configurations described herein allow for a variety of sampling modes. For example, studies showed that when operating in a weekly autonomous mode at 1 sample per hour for a week, the sensor devices used 96% of the energy available from the thin-film battery, a 2 uAh thin-film battery. When operating in a daily autonomous mode at 1 sample per 6 minutes for a day, the device used up 29% of the energy available in the same thin film battery.

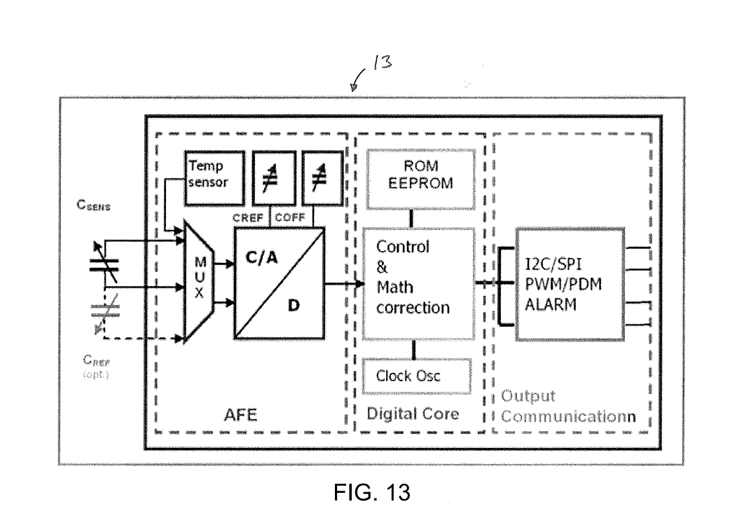

FIG. 13 illustrates a schematic of the logic configuration of the control/processing unit 13 that controls receiving measuring data from the pressure sensor (C_{SENS}), storing the pressure measurement data and optionally processing the pressure measurement data and controlling communication output of the data to the external device. In embodiments including the optional reference sensor 15 (C_{REF}), the control/processing unit controls receiving of measurement data from the reference sensor and optionally one or more other data sources (e.g. temperature sensor) then stores the reference data associated with pressure measurements or utilizes the data to process the measurement data before transmission of data to the external device or base station. One advantageous aspect of the vertically stacked design of the sensor device is that all electrical connections to the pressure sensor and the optional reference sensor that reach the ASIC input stage can be provided on the backside of the wafer. The device can be configured such that a reference plate associated with the reference sensor is fully isolated from the outside of the sensor device such that only the sensing plate is in contact with the media being sense (e.g. aqueous humor, vitreous body or cerebral fluid depending on the application or targeted area in which the sensor device is used).

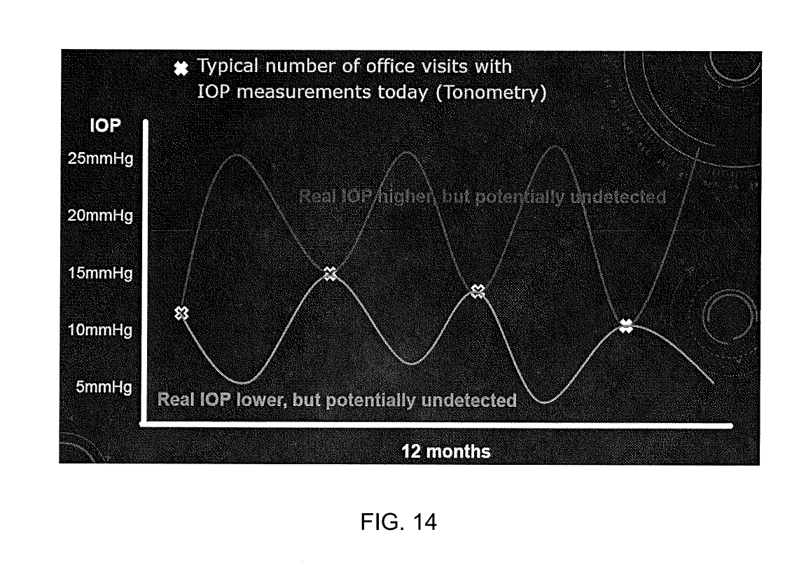

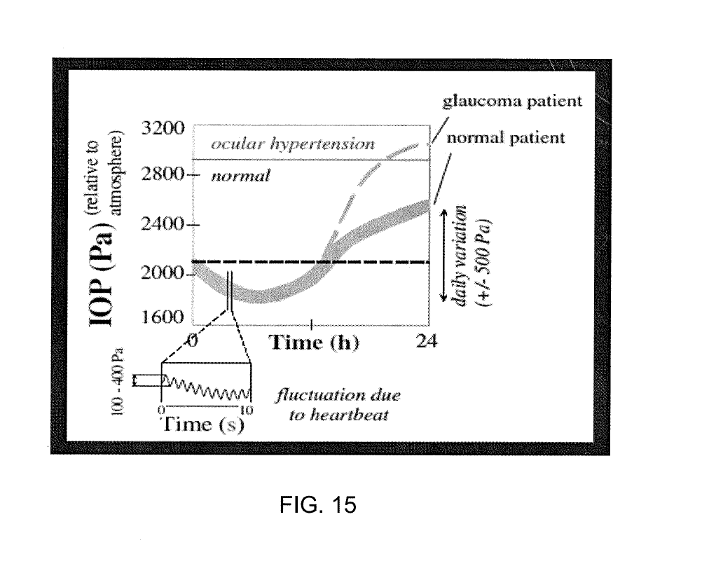

FIG. 14 illustrates variations that may occur in a patient's IOP and potentially undetected peaks in IOP that may occur outside of the typical number of office visits a glaucoma patient would experience in conventional IOP monitoring. As can be seen, infrequent pressure monitoring does not provide an accurate depiction of the range of IOP experience by a glaucoma patient. Increases in pressure outside the discrete monitoring visits may cause damage to the optic nerve and result in irreversible loss of vision. FIG. 15 illustrates the variations in IOP that may occur during a single 24-hour period. As can be seen, glaucoma patient may experience fluctuations in IOP considerably greater than those of a normal patient. In addition, measurement of IOP at a given moment may be further affected by various factors, such as heartbeat or elevation, that may change throughout the day. By providing a miniature device that can obtain multiple measurements of IOP each day, up to one sample per hour, during an extended time increment of at least one week, the methods of the present invention allow for vastly improved monitoring of IOP with minimal patient interaction beyond period charging of the device with a personal handheld device, such as a smart-phone.

The present methods may also provide improved monitoring by allowing for adaptable sampling. For example, the sensor device may automatically adjust sampling rates in response to a detected patient condition (e.g. activity, sleep, elevated IOP) or the sampling rates may be adjusted by a physician according to a particular sampling protocol prescribed by the physician and uploaded to the device upon the next re-charging and/or data acquisition with the external device. In one aspect, the sampling program includes at least a first sampling rate and a differing second sampling rate. Either of the sampling rates may be a fixed or various sampling rates. The sampling rate may be selected in response to a measured physiological condition, such as a pressure measurement exceeding a pre-determined IOP threshold. For example, the first sampling rate may be sampling every three hours and upon detection of an elevated IOP, the sensor device samples at a second higher rate, such as every hour or every half-hour, until the increase in measured IOP is resolved. In another aspect, the measured physiologic condition may be waking hours of the patient. Upon detection of eye movements indicative of waking hours or optical detection of light associated with the patient's waking hours, the sensor device may sample at a higher rate than when the patient is sleeping.

IOP Monitoring System Use Models

In one aspect, the sensor device may be configured with various modes of operation for different uses. Examples of use models for use with a monitoring system in accordance with embodiments of the invention are shown below in Table 1.

TABLE 1
Monitoring System Use Modes

| Mode Description | Feature (a) | Feature (b) | Comments |
|---|---|---|---|
| 1. Factory | Unique 64 bit ID | Calibration | Read only data |
| initialization (Unique | | coefficients | |
| ID and calibration | | | |
| coefficients) | | | |
| 2. Sampling Mode (a) | Single data point | Data streaming | Time window limited such as |
| real-time | (GAT comparison | (20 ms to 1 | Operating Room procedure |
| | mode) | minute) | |
| 3. Sampling Mode (b) | Autonomous | Non-autonomous | Sampling window (1 day to |
| baseline | (1 m to 1 hour) | (1 s to 1 minute) | multi-day) - therapy |
| | | | management |
| 4. Variable data | Multi-sample rate | Multi-period | Ophthalmologist to define |
| acquisition profile | | | variable acquisition |
| | | | sequences |
| 5. IOP data | Raw data pre- | raw data in | IOP monitoring has preset |
| processing options | processing option | receiver | pre and post processing. |
| | in implant with | accessible for | Customization options |
| | MCU firmware | custom post- | available by firmware |
| | | processing in | (implant) and software in |
| | | Apps | Apps/receiver |
| 6. Data integrity | Data processing | Data received | Outliers can be readily |
| | option | monitored within | identified and omitted from |
| | | acceptable range | trend analysis |
| 7. Alert modes | Mean shift | Fluctuation rate | Data curation managed in |
| | Min, Max | | receiver (ECC, out of range |
| | | | data) |
| 8a. Recharge mode | Maintain | Data extracted | Incremental mode option |
| (data ready) | minimum power | | |
| | charge | | |
| 8b. Exception modes | Battery low | Deep sleep wake | Preclude acquisition to |
| | voltage (disabled | up mode | protect TF battery |
| | device) | | |
| 9. Patient therapy | Reoccurring | Compliance | Mapping of drug application |
| management (e.g. | event | monitoring (IOP | in calendar |
| drug schedule) | (x hours, y days) | reduction) | |

Each of the above use models is described in further detail as follows:

1—Unique ID and Calibration Coefficients:

In some embodiments, this mode is defined as a 64 bit word that assign a number to each implant to recognize each implant individually. This register is read by the external reader at the start of any session with an implant to identify the patient and also associated the calibration coefficients specific to the unit (IOP-connect).

2—Sampling Mode (a) Real-Time:

a. In this mode the sampling is either on demand (query of one IOP reading which includes one absolute pressure reading, temperature and battery voltage.
b. For sampling of 20 ms period (50 Hz) to 1 minute, the data is query (streaming mode) at the faster rate and typically limited for a shorter period of time (less than 30 minutes). This option is very demanding on the battery and either full charge or memory size will limit the period of sampling acceptable. In this mode, the device might require a recharge in between each sampling period which could be of 20 minutes or more. The battery charge can be managed to stay between 25 and 75% to maintain its operation.
c. For both modes, the device is connected to a receiver and operating in non-autonomous mode. In these modes, higher frequency events can be captured and real-time monitoring enabled.

3—Sampling Mode (b) Baseline:

a. Autonomous mode: the sampling rate can be set between 1 minute and 1 hour. In this case, the sampling window is limited between period that matches the memory and charge available. If a period requested is longer than the capacity of the device, the reader application will segment the sampling window in sub-period and interlace a power/data cycle to download the device memory and recharge the battery. This option is transparent to the patient and practitioner. The sampling period can be defined with different rate and different period length. For example, (1 minute/1 day), (1 hour/3 days), (1 minute/1 day), (1 hour/6 days), etc. . . . This sequence is managed by the receiver near the patient without any intervention. Some sequences might not be continuous and require the insertion of recharge
b. Partially-Autonomous mode: for sampling rate of 1 s to 1 m, the period is limited in the same way as defined above (2.a) but the battery and/or memory will limit the acquisition period. At the end of the period of acquisition, the reader can be required to reconnect with implant and collect information coupled with a recharge. In this mode the acquisition is autonomous compare to 2.b which is manage in streaming mode (non-autonomous).
c. For mode 3 (a/b), we are using this mode for therapy management to define the effectiveness of the drug regimen against the baseline. Latency of the drug can be monitored and characterized. Longer term trend captured and characterized. Higher frequency events are typically not captured within 1 minute. For sampling above 1 sample/minute, the objective is to extract the baseline of a patient IOP.

4—Variable Data Acquisition Profile:

a. In the period mode 3, we have generalized the acquisition to a sequence of (rate/time window) that are repeated at various rates/periods. Due to the unique programmability of the device and the ability to capture different events/profiles; the sequence are broadly definable and only limited by the recharging or data downloading cycle. Some limitations have to be taken into account (size of samples set), length of sampling limitation due to the charge of the battery.

5-IOP Data Processing Options

a. Due to the availability of a MCU (microcontroller) within the IOP-connect implant, the device is capable of pre-processing the data. Multiple DSP functions are available and are coordinated with the receiver. The data sample (absolute pressure) needs to be combined with atmospheric pressure to calculate gauge pressure. If a pre-processing sequence is requested with the implant, the same pre-processing can be replicated inside the receiver before generating the final dataset.

i. For example, the IOP-connect will collect 8 samples and average them to eliminate the variation due to the cardiac activity (ocular pulse amplitude).

b. For post-processing, this is done within the receiver or the apps.

6-Data Integrity—for all Data Processing and Sampling, the Receiver Will Monitor the data within acceptable range. In some embodiments, no data is deleted but rather it is flagged for potential inconsistency.

a. The domain of data curation is supported across different layer of data analysis and screening. The practitioners will have access to graphic display of historical data which will have also statistical significance over extended period of time (years/months etc.).
b. Data integrity and privacy is maintained with database management services that are implemented across the dataflow from the patients to any consumer of data. Meta data will also be added to support a broad range of services. As an example, firmware can be configured so that pharmaceuticals or researchers do not have access to patient identity associated with transmitted data.
c. As an example, if the receiver pressure sensor wasn't located near the patient's head, it is possible that the atmospheric pressure captured has an offset and generating unusable data. Potential data correction algorithms could be applied and field testing completed to identify a broad range of scenarios. The flexibility of the system should be capable of generating complex functions and verification modes. Statistical parameters and other data set characteristics could be used to support a broad range of analysis. Data analytics can be applied over time to perform data mining on IOP to identify pattern, correlation with events, identify specific components in the signal recorded (spectral decomposition).

7—Alert Modes:

these modes will present to the practitioner a broad range of event detections that are either built-in or user-defined. A library of alerts are available and new options uploaded to each receiver when they are connected to InjectSense server.

a. Event type: min, max, fluctuation (increase/decrease), spikes, data error, etc. . . . it is obviously limited by the data set logged. In some cases, if a particular event is detected (large fluctuation), it will possible to generate a different sampling (rate/window) which will try to record additional information between samples. This dynamic adjustability although very powerful may be limited by the memory and power available. In some case, the alert might not be able to adjust the sampling sequence due to the state of the implant. More historical data will have to be generated to assess in which conditions such adjustment can be made. The possible adjustment on the sampling mode will also have to be defined across a different type of alerts. This adjustment might be inserted into a sampling sequence and after completion of this new sequence, the previously defined sampling sequence can be applied.

8a/8b—Recharge Mode and Battery Management/Exception Modes:

a. Due to the specific characteristics of the battery (LiPON), the battery management can be incorporated within the implant and also within the receiver to maximize lifetime and avoid degradation of the initial performance. InjectSense will implement a conservative approach that alleviates potential failure modes.
b. With the option of using SRAM which requires continuous powering, the battery will be managed between 25 and 75% of the charge. The receiver has a continuous log of all activities of the IOP and predict some conditions that will require establishing a link with the implant. Either the receiver or the Apps (smartphone) will inform the patient of a specific action required.
c. The implant device IOP-connect has building monitoring that will protect it from potential failure (e.g. full drainage of battery), over-sampling, etc. . . . .
d. Other diagnostics are available to assess the device operating conditions and potential issues that will need some form of intervention by the receiver. These diagnostics will typically be running with a link established with the external receiver and not during an autonomous mode to preserve the battery charge.
e. If the device is reaching the limit of the battery, the device can be forced into deep sleep including the RTC (temporary kill mode). In this case, the device is locked and will require an office visit with the practitioner to unlock the device.

9—Patient Therapy Management:

a. One important benefit of IOP connect continuous monitoring is to provide a detailed picture of the effectiveness of the drug regimen. Capturing the drug latency, duration of IOP reduction, quantify the effectiveness dynamically will provide a clear picture of the effect (or lack of) for the drug. Adjustment of the dose and incremental effect can be captured and quantified. Other factors like patient lifestyle and how other parameters affecting IOP are managed can be potentially identified (patient motion, position and in general activities) with detailed correlation with IOP trend/fluctuations.
b. Other parameters like respiration rate, blood pressure fluctuation and other physiological aspects can be correlated with IOP fluctuations to establish cause-effect relationships that could prove an influential in the patient health and in particular preventing the progression of glaucoma towards blindness.
c. Big data paradigms open to analytics that allow, across extended period of time, for a single patient or across large population of patients to better understand how therapy effectiveness and influential factors will need to be controlled.
d. The continuous monitoring will also allow diurnal and nocturnal acquisition to potential establish if circadian cycle exist within IOP or not.
e. Personalized treatment/therapy can be selected between accurate and continuous monitoring of IOP for years with patients and correlate with glaucoma diagnostics like Perimetry and disk/cup ratio for the optical nerve head.
f. Remote monitoring of patient and remote configurability will allow a more effective and cost effective relationship between patients and ophthalmologists by reducing the number of visits to the minimum required and concentrate the therapy on unique dataset for each patient filling the gap previously created with Goldman applanation tonometry (GAT) which is only in-office procedure and generate very limited data to one sample.

Advanced Power Management

In one aspect, the ASIC include a supercapacitor and a thin-film battery along with an advanced power management system utilizing a microcontroller. The power management system serves to: (a) manage the power transfer from the battery to the supercapacitor due to the high impedance limiting factor of the battery. The supercapacitor is receiving energy needed for each block and switched back and forth with the battery to perform impedance conversion. Using directly the battery is not viable due to its high impedance seen at the connectors (e.g. 40 k Ohms); and (b) operate such that the supercapacitor acts as a fast charging energy source that is decoupling the battery from the circuit blocks and provides regulated/stable supply voltage with load management (e.g. adjusting energy per cycle).

In another aspect, the ASIC has been upgraded to a top level state machine with a real time clock (RTC) coupled with a microcontroller (MCU). This configuration provides increased flexibility for the configurability of the circuit blocks. This configuration allows improved configurability and programmability of the implant with firmware/software as compared to a hardwired state machine. This configuration also allows firmware updates to be applied to the implant, support for a broader range of use models, and adjustable performance parameters either by reprogramming firmware or enable dynamic (re)configurability of the IOP measurement with different sampling rate/time window depending on the data received by the transducer. As an example, when using a sensor device for measuring IOP, if the IOP is very stable, the sampling rate can be reduced and vice versa. This adaptive mode can be made available to the user or can be configured within the firmware of the MCU.

In yet another aspect, the described sensor device configuration provides energy management layered between static and dynamic power usage. For example, in the embodiment described above, the supercapacitor is used as a dynamic power source and the battery is used as the power reserve addressing both static and dynamic energy usage.

In regard to transmitting power to the device, the induced voltage required to sufficiently charge the implanted device is determined as follows:

Assuming an uncontrolled exposure limit of 1 mW/cm2 over a 30 minute period, the following equation (1) may be used to determine the induced voltage required to charge the implanted sensor device.

$$\frac{{\vert E \vert}^{2}}{2 η} = \frac{{10}^{- 3}}{{10}^{- 4}} ⇒ \vert E \vert = 86.83 V \text{/} in . \qquad ( 1 )$$

It is assumed that the implant makes an a 45 degree angle with the plane of the incident field during charging with an external charging device. The external charging device may be incorporated into a pair of glasses worn by the patient or other such device that can be worn by the patient for a duration of time sufficient to charge the implanted sensor device, generally less than three hours, typically between about 15 minutes and 3 hours, more typically between about 20 minutes and 2 hours. In some embodiments, charging duration is between about 20 minutes to 70 minutes after 1000 recharge cycles.

In determining the induced voltage required, the effective length of the dipole=2 mm. Using equation (2) yields:

2 mm/\sqrt{2}=1.414 mm. (2)

Thus, the O.C. voltage at the implant is determined by the following equation (3):

Voc=|E1lett=86.85×1.414 l⁻³=0.123√ (3)

Capacityance of a short dipole is determined according to the following equation (4):

$$C = \vert \frac{\text{?}}{ln ( h / a )} = \frac{π \times 8.8542 \times {10}^{- 12} \times 2 \times {10}^{- 3}}{ln ( \frac{2}{0.2} )} = 24.16 fF \\ \text{?} \text{indicates text missing or illegible when filed} \qquad ( 4 )$$

such that capacitance of the rectifier stage≈100 fF to 500 fF (see diagram below).

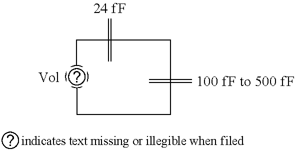

The voltages across the diode≈100 mV. Matching may be used in order to meet the threshold voltage of diodes.

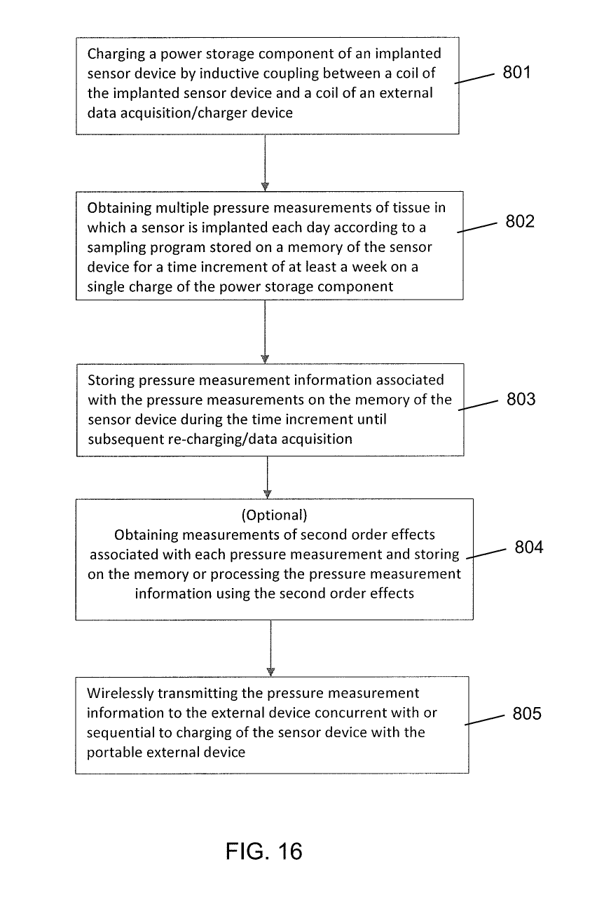

FIG. 16 is a flow chart that illustrates an example method in accordance with aspects of the invention. The method includes steps of: charging a power storage component of an implanted sensor device by inductive coupling between a coil of the implanted sensor device and a coil of an external data acquisition/charger device 801; obtaining multiple pressure measurements of tissue in which a sensor is implanted each day according to a sampling program stored on a memory of the sensor device for a time increment of at least a week on a single charge of the power storage component 802; storing pressure measurement information associated with the pressure measurements on the memory of the sensor device during the time increment until subsequent re-charging/data acquisition by the external device 803; optionally obtaining measurements of second order effects associated with each pressure measurement and storing on the memory or processing the pressure measurement information using the second order effects 804; and wirelessly transmitting the pressure measurement information to the external device concurrent with or sequential to charging of the sensor device with the portable external device 805. In one aspect, the sensor device operates for a time increment of at least one week on a single charge, preferably two or three weeks on a single charge.

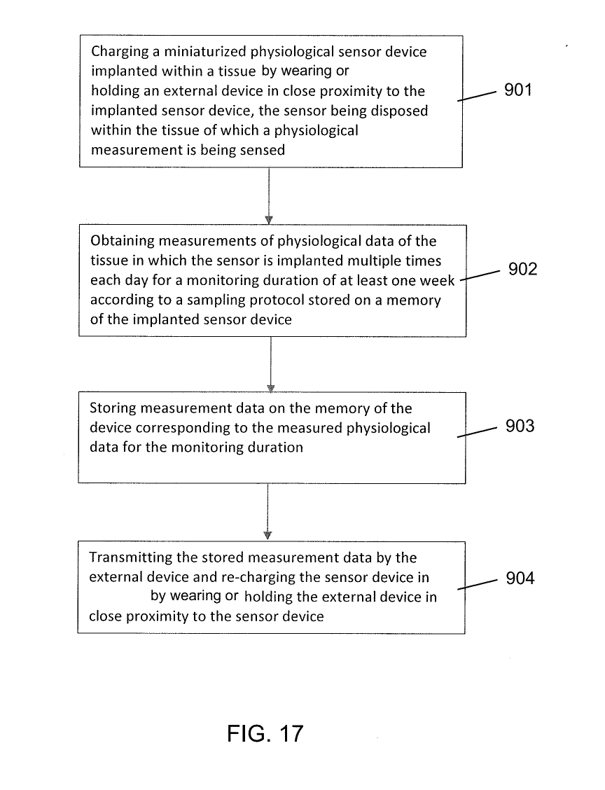

FIG. 17 is a flow chart that illustrates an example method in accordance with aspects of the invention. The method includes steps of: charging a miniaturized physiological sensor device implanted within a tissue by holding or wearing an external device in close proximity to the implanted sensor device, the sensor being disposed within the tissue of which a physiological measurement is being sensed 901; obtaining measurements of physiological data of the tissue in which the sensor is implanted multiple times each day for a monitoring duration of at least one week according to a sampling protocol stored on a memory of the implanted sensor device 902; storing measurement data on the memory of the device corresponding to the measured physiological data for the monitoring duration 903; and transmitting the stored measurement data by the external device and re-charging the sensor device in less than 10 second by holding the external device in close proximity to the sensor device 904. It is appreciated that the methods shown in FIGS. 9 and 10 are illustrative and that various steps may be modified and still remain in keeping with the advantageous aspects of the invention described herein.

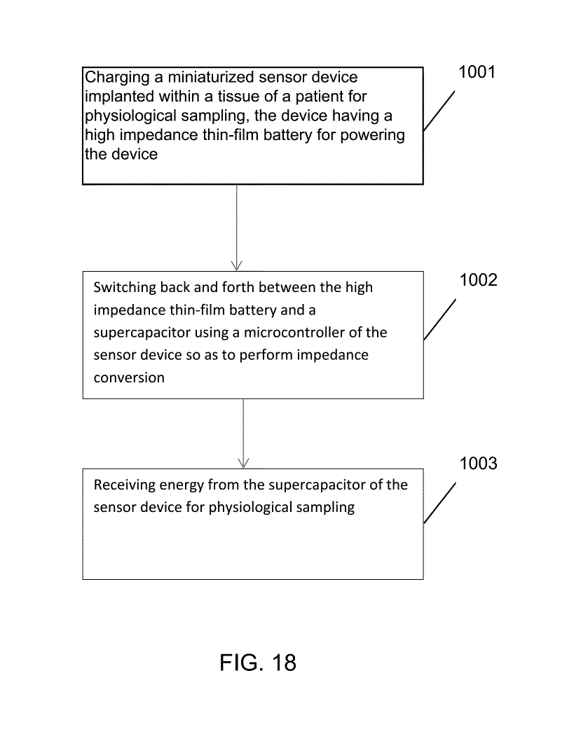

FIG. 18 is a flow chart that illustrates an example method of powering a miniature implanted sample device in accordance with aspects of the invention. The method includes steps of: charging a miniaturized sensor device implanted within a tissue of a patient by use of a high impedance thin-film battery of the device 1001; switching back and forth between the high impedance thin-film battery and a supercapacitor using a microcontroller of the sensor device so as to perform impedance conversion 1002; and receiving energy from the supercapacitor of the sensor device for physiological sampling 1003.

In the foregoing specification, the invention is described with reference to specific embodiments thereof, but those skilled in the art will recognize that the invention is not limited thereto. Various features and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. It is recognized that the terms “comprising,” “including,” and “having,” as used herein, are specifically intended to be read as open-ended terms of art.

What is claimed is:

1. A telemetry method for monitoring IOP, the method comprising:
obtaining a plurality of IOP measurements IOP within a vitreous body of an eye of a patient with a sensor device implanted within the vitreous body, wherein the plurality of pressure measurements are obtained over a monitoring period and powered by an energy storage component of the implantable sensor device;
storing IOP data corresponding to the plurality of pressure measurements on a recordable memory of the implantable sensor device for at least the monitoring period; and
wirelessly transmitting the IOP data from the implantable sensor device to an external device when the external device is in proximity to the implantable sensor device.

2. The method, wherein the plurality of pressure measurements are obtained according to a sampling program stored on the memory of the implantable sensor device.

3. The method of claim 1, further comprising:
processing the plurality of pressure measurements with a processor of the implanted sensor device such that the IOP information corresponds to a trend or variation in IOP over the time increment.

4. The method of claim 1, further comprising:
obtaining measurements of second order effects associated with the plurality of pressure measurements with a reference sensor of the sensor device.

5. The method of claim 4, further comprising:
processing the plurality of pressure measurements to account for the second order effects associated with the plurality of pressure measurements with the reference sensor.

6. The method of claim 1, wherein obtaining the plurality of pressure measurements comprises measuring pressure within the vitreous body with a sensing membrane of the pressure sensor disposed entirely within the vitreous body.

7. The method of claim 1, wherein obtaining the plurality of pressure measurements comprises measuring pressure with the pressure sensor of the sensor multiple times each day during the time increment of at least a week.

8. The method of claim 7, wherein obtaining the plurality of pressure measurements according to the sampling program comprises measuring pressure with the pressure sensor up to every hour during the time increment.

9. The method of claim 7, wherein obtaining the plurality of pressure measurements according to the sampling program comprises measuring pressure with the pressure sensor at regular sampling intervals, the regular sampling interval within a range of 5 minutes and 2 hours.

10. The method of claim 1, further comprising:
switching between differing use modes of the sensor device by use of a microcontroller, wherein the differing use modes include any of: a factory initialization mode, a real-time sampling mode, a baseline sampling mode, a variable data acquisition profile mode, an IOP data processing mode, a data verification mode, an alert mode, a recharge mode, an exception mode, and a patient therapy mode.

11. The method of claim 1, wherein obtaining the plurality of pressure measurements comprises measuring pressure with the pressure sensor at a first sampling rate, wherein the first sampling rate is a fixed sampling rate.

12. The method of claim 11, wherein obtaining the plurality of pressure measurements comprises measuring pressure with the pressure sensor at a second sampling rate based on one or more detected conditions.

13. The method of claim 12, wherein the second sampling rate is higher than the first sampling rate and the physiological condition is a measured IOP exceeding a pre-determined threshold.

14. The method of claim 12, wherein the second sampling rate is a variable rate based on the one or more conditions.

15. The method of claim 12, wherein the one or more conditions includes waking hours of the patient.

16. The method of claim 15, wherein the second sampling rate comprises sampling every hour during waking hours.

17. The method of claim 1, wherein obtaining the plurality of pressure measurements during the time increment is powered by energy stored in the energy storage component of the sensor device received in a single charging of the energy storage component, the time increment being at least one week.

18. The method of claim 1, further comprising:
wirelessly receiving data associated with the sampling program from the external device and updating the sampling program.

19. The method of claim 1, wherein wirelessly transmitting the IOP data is carried out by one or more coils of the sensor device and a corresponding coil of the external device.

20. The method of claim 1, further comprising:
charging the energy storage component by inductive coupling between one or more coils of the implantable sensor device with a corresponding coil of the external device.

21. The method of claim 20, wherein wirelessly transmitting the IOP information to the external device is performed concurrently or sequentially with receiving charging energy.

22. The method of claim 1, further comprising:
wirelessly receiving energy from the external device to charge the sensor device by storing the wirelessly received energy in the energy storage component of the sensor device while implanted.

23. The method of claim 22, wherein wirelessly receiving charging energy comprises inducing a voltage on a receiving coil of the sensor device with a corresponding coil of an external device magnetically coupled with the receiving coil.

24. The method of claim 23, wherein the voltage induced in the receiving coil is regulated by a voltage regular and a rectifier so as to provide a stable power supply to the implanted sensor device.

25. The method of claim 23, wherein the sensor device includes a decoupling capacitor configured to store sufficient energy from the voltage induced in the receiving coil to operate the sensor device for a duration of at least one week.

26. The method of claim 23, wherein operating the sensor device consumes about 1 μWatt of power or less during measuring and storing of pressure measurement data such that the sensor device can operate for a duration of at least one week before recharging.

27. The method of claim 19, further comprising:
transitioning into a sleep mode consuming about 1 nW of power or less during periods of time outside of obtaining the plurality of pressure measurements, transmitting data and wirelessly receiving charging energy.

28. The method of claim 19, wherein the device sends and receives data associated with the pressure measurements and receiving energy to power the device according to a passive RFID configuration upon receiving RF energy transmitted by the external device.

29. A method of calibrating an implantable pressure sensor device, the method comprising:
obtaining a plurality of pressure measurements with the implantable sensor device under controlled conditions at differing values of one or more controlled parameters;
determining variations between the plurality of pressure measurements at the differing values of the one or more controlled parameters, wherein the variations correspond to mechanical characteristics affecting the plurality of pressure measurements that are particular to the implantable sensor device; and
storing calibration data associated with the determined variations in a memory of the implantable sensor device for use in adjusting in-situ measurements obtained from the sensor device while implanted to improve accuracy of plurality of pressure measurements.

30. The method of claim 29, further comprising:
storing the calibration data with a unique identifier associated with the implantable sensor device such that an external device communicatively coupled with the sensor device while implanted receives the stored calibration data for use in processing the plurality of measurements received from the device having the unique identifier.

31. The method of claim 29, wherein the implantable sensor device comprises an IOP sensor, the plurality of measurements comprise a plurality of pressure measurements and the one or more controlled parameters comprise a pressure and/or a temperature.

32. An implantable sensor device for measuring IOP of an eye of a patient, the device comprising:
a pressure sensor adapted for measuring a plurality of pressure measurements, wherein the pressure sensor is configured such that a pressure sensing membrane of the pressure sensor is disposed entirely within a vitreous body of the eye;
a control unit coupled to the pressure sensor and comprising a processor configured to control sampling of pressure measurements with the pressure sensor according to a sampling program;
an energy storage component coupled to the control unit and configured to wirelessly receive energy while implanted sufficient to power sampling and storage of the plurality of pressure measurements for a time increment of an extended monitoring period; and
one or more coils adapted to wirelessly receive energy for charging of the energy storage component and to wirelessly transmit and receive data associated with the plurality of pressure measurements.

33. The sensor device of claim 32, wherein the control unit is configured to:
initiate wireless communication with an external device for wireless communication and/or receiving of charging energy upon detection of the external device in proximity to the implanted sensor device;
perform charging and wireless communication concurrently or sequentially when the external device is in proximity to the implanted device; and/or
optimize wireless charging and/or wireless communication based on a detected distance between the external device and the implanted sensor device.

34. The sensor device of claim 32 wherein the sensor device comprises a chip-scale package formed, at least in part, on a wafer or rigid substrate, wherein the one or more coils are coiled in-plane with the sensor device.

35. The sensor device of claim 32, wherein the implantable sensor device is configured so as to obtain multiple pressure measurements each day for a time increment of at least one week powered by the energy stored in a thin-film battery from a single charging and store IOP information associated with the plurality of pressure measurements for the time increment,
wherein the sensor device is configured to perform impedance conversion by switching back and forth between a supercapacitor and the thin-film battery such that energy for obtaining multiple pressure measurements is received from the supercapacitor.
